(19) United States
(12) Patent Application Publication
(10) Pub. No.: US 2026/0133162 A1
(43) Pub. Date: May 14, 2026

(54) METHODS AND DEVICES FOR MOLECULAR CHARACTERIZATION

(71) Applicant: Grolltex, Inc., San Diego, CA (US)

(72) Inventors: Aliaksandr Victorovich Zaretski, San Diego, CA (US); John M. Lyle, San Diego, CA (US); Stephen Robert Fairclough, San Diego, CA (US)

(21) Appl. No.: 19/444,223

(22) Filed: Jan. 9, 2026

Related U.S. Application Data
(63) Continuation-in-part of application No. PCT/US25/12246, filed on Jan. 18, 2025.
(60) Provisional application No. 63/623,148, filed on Jan. 19, 2024.

(51) Int. Cl. G01N 27/447; C12Q 1/6869

(52) U.S. Cl. CPC G01N 27/44791; C12Q 1/6869; G01N 27/4473

(57) ABSTRACT

A device for characterization of polymers is described. The device includes a plurality of atomically thin electrodes with their edges exposed along a fluidic channel through which a single stranded polymer molecule is controllably positioned. The polymer includes a series of subunits where each in sequence is identified by an electrical signal between two of the electrodes when in contact with the two electrodes. The position of the polymer strand within the fluidic channel is controlled by voltages applied to the atomically thin electrodes as well as global electrodes located within the fluidic channel. The electrical signal includes tunneling currents along the length of each segment, ionic currents within solutions containing the polymer segments, and, in some embodiments tunneling currents across each polymer segment, and, based upon those measurements, identifying the chemical composition of each segment and therefore the chemical composition of the polymer as a sequence of subunits.

### CROSS REFERENCE TO RELATED APPLICATIONS

This application claims priority to U.S. Application PCT/US25/12246 filed Jan. 19, 2024 and U.S. Provisional Application 63/623,148 filed Jan. 18, 2025 both with the same title and same inventors and currently pending.

### BACKGROUND

### Field

The present disclosure relates generally to the fields of biology and chemistry, and in particular analysis of molecular structures by identifying and determining the sequence of subunits of polymer chains.

### Description of the Related Art

A number of conventional techniques are available for characterizing biomolecules, such as nanopore sequencing, which is used to analyze biological molecules by observing a signal related to molecular passage through the nanopore. Commercially available nanopore sequencing devices rely on a protein to comprise the nanopore and in some cases to regulate the speed of translocation through the nanopore. This results in a long stretch (e.g., 8+) of nucleotides or other monomers being present in the nanopore and contributing to the electrical signal across the nanopore at any time. Further, the motive force which drives the passage is usually hydrostatic and constant and distinct from the makeup of the polymer being analyzed, resulting in stochastic rates of passage through the nanopore. These two fundamental problems combined result in a signal that requires substantial deconvolution and with that, errors in the determination of the nucleic acid, amino acid, or other polymer sequence. Such devices and methods however suffer from low signal to noise, insufficient translocation speed control, manufacturability, and other shortcomings. Typical prior art examples include US patent publication 2014/0125310 from Samsung Electronics, US 2015/0377830 from IBM, US 2002/0023684 from Caliper Technologies Corp., and, U.S. Pat. No. 5,622,872 from Biocircuits Corporation. All four prior art examples suffer variously from the deficiencies as listed. For example, application 2014/0125310 requires construction of a nanopore structure with dimensions at current manufacturing limits of about 1 nm or less and uses fluid dynamics to control movement of a target polymer molecule through the nanopore. Movement of a target molecule and lack of control thereof limits time available for measurement of currents and fields across the target molecule, limiting signal to noise and ability to identify monomer units within the target polymer. Prior art devices are limited to measure currents and fields transverse to the long axis of the polymer chain, limiting signal to noise and specificity to identify subunits. The other references all suffer from the same or similar deficiencies. There exists a need for developing systems and methods capable of exerting precise control over biomolecule translocation speed and direction and exhibiting sufficient specificity and signal to noise of measurement. There is a need for polymer characterization systems and methods that measure currents and fields along the length of a polymer backbone. Such systems should not have shortcomings arising from the use of biology-derived components or the necessity to create patterned features smaller in dimension than allowed by current photolithography standards.

### BRIEF DESCRIPTION OF THE INVENTION

Disclosed herein include sensors for characterizing a stranded molecule. In some embodiments, the sensor comprises: at least one first electrode sheet, at least one second electrode sheet, and at least one first insulating layer disposed between the at least one first electrode sheet and the at least one second electrode sheet, wherein the sensor comprises a sensor edge capable of interacting with a stranded molecule, thereby modulating a tunneling current between the at least one first electrode sheet and the at least one second electrode sheet and along the length of the polymer chain. Electrical measurements can be made both along the length of a limited number of monomer units and transverse to the long axis of the polymer. The invented device and methods include the ability to control movement of the target polymer along the sensor edge such that measurements of tunneling current can be made over an extended time to increase signal to noise and improve specificity of identifying particular monomer units and improve accuracy of sequence determination of, for example, base units in a nucleic acid polymer.

In some embodiments, the at least one first electrode sheet and/or the at least one second electrode sheet is atomically thin. In some embodiments, the at least one first electrode sheet and/or the at least one second electrode sheet comprises a two-dimensional electrically conductive material. In some embodiments, the two-dimensional electrically conductive material comprises gold, titanium nitride (TiN), poly(3,4ethylenedioxythiophene) polystyrene sulfonate (PEDOT: PSS), molybdenum disulfide (MoS₂), copper, platinum, nickel, or, graphene, or a combination thereof. In some embodiments, the at least one first electrode sheet and the at least one second electrode sheet comprise a same electrically conductive material or different electrically conductive materials. The at least one first electrode sheet, the at least one second electrode sheet, and the at least one first insulating layer have adjacent exposed edge portions which collectively form the sensor edge. In some embodiments, there are one, two, three, four, or more sheets of electrodes and corresponding interleaved multiple sheets of insulating layers separating the one or multiple electrodes.

The at least one insulting layer comprises ceramics, diamond, two-dimensional polymers, self-assembled monolayers or bilayers, and/or other electrically insulating organic and inorganic moieties. In some embodiments, the ceramics comprises Si₃N₄, Al₂O₃, HfO₂, or a combination thereof. In some embodiments, the at least one first insulating layer comprises one or more atomically thin sheets. In a preferred embodiment, the insulating layer comprises boron nitride or hexagonal boron nitride (hBN). In some embodiments, the insulating layer comprises multiple sheets where the number of sheets in an insulating layers determines the spacing between adjacent electrode sheets. The sensor edge can be configured to partially or fully surround the stranded molecule.

In some embodiments, the sensor edge is functionalized with one or more chemical group(s). In some embodiments, the functionalization comprises hydrogenation, hydroxylation, carboxylation, carbonylation, amination, or a combination thereof. In some embodiments, the sensor edge chemically modified to minimize the tunneling electrical resistance between the sensor edge and along the stranded molecule, e.g., configured with a charge-injecting group. In some embodiments, the sensor edge is configured to enhance the tunneling electrical interaction between the sensor edge and the stranded molecule. In some embodiments, the configuration enhances binding with the stranded molecule through hydrogen bond interaction, electrostatic interaction, hydrophobic/intercalating interaction, biomolecular binding, or a combination thereof. In preferred embodiments, the sensor edge is functionalized with a p-phenylenediamine moiety. In some embodiments, the edge portions of the electrode sheet and the insulating layer are functionalized with a same functional group or different functional groups. A voltage drop or current between a first electrode sheet and a second electrode sheet is measured when the stranded molecule is adhered to the sensor edge and a voltage is applied across the electrode sheets.

In some embodiments, the sensor device further comprises a fluidic passage disposed within a support structure (e.g., a support structure comprising a semiconductor material), two or more global electrodes capable of providing a voltage to translocate the stranded molecule electrophoretically through the fluidic passage, at least one edge sensor disposed in the support structure with the sensor edge exposed to the fluidic passage; and a detector configured to detect a current between the at least one first electrode sheet and the at least one second electrode sheet as the stranded molecule is adhered to the sensor edge.

In some embodiments, the fluidic passage comprises a vertical fluidic passage section and the at least one sensor is disposed in the support structure with the sensor edge exposed to the vertical fluidic passage section. In some embodiments, the sensor edge is in a closed-shape geometry or a linear geometry forming one or more apertures in the support structure. In some embodiments, the fluidic passage comprises a lateral fluidic passage section in fluidic connection with the vertical fluidic passage section and wherein the at least one sensor is disposed in a bottom of the lateral fluidic passage section with the sensor edge exposed to the vertical fluidic passage section. In some embodiments, the lateral fluidic passage section and/or the vertical fluidic passage section has a dimension configured to accommodate no more than one stranded molecule.

In some embodiments, the lateral fluidic passage section has a wedge shape. In some embodiments, the sensor device is a sensor chip comprising multiple edge sensor devices. In some embodiments, the sensor device comprises at least two, three, four, or five sensors. In some embodiments, each sensor is exposed to a distinct fluidic passage. In some embodiments, the sensor device comprises: a loading well and a fluidic reservoir both in fluidic connection to the fluidic passage, wherein the loading well provides an inlet to the fluidic passage and the fluidic reservoir provides an outlet from the fluidic passage.

Also disclosed are methods for using the sensor device. In some embodiments, the method comprises applying a voltage across the fluidic passage to produce a current flow through the fluidic passage, causing a single stranded molecule to translocate through the fluidic passage at a time and allowing said single stranded molecule to interact with the sensor edge of the at least one sensor; detecting a first electrical signal between the at least one first electrode sheet and the at least one second electrode sheet of the sensor edge when a first portion of the stranded molecule is adhered to the edge portions of the at least one first electrode sheet and the at least one second electrode sheet of the at least one sensor; and determining a characteristic of the first portion of the stranded molecule based on the detected first electrical signal. Unlike prior art sequencing techniques that require purification or preparing chemical derivatives of the biomolecule to be sequenced, the invented single molecule approach of the current invention can produce a signal with sufficient specificity such that, with training, it can distinguish each of the polymers subunits passing across the edge to a sufficient degree to assign its identity to a particular class of subunit and then proceed with identification of the subunit sequence of that biomolecule. The device and methods are applicable to a variety of biopolymers and other polymer types such as DNA, RNA, protein, other biomolecules and non-biological polymers. Thus, a cell lysate or other biospecimen (e.g., plasma) can be loaded directly onto the device and generate sequence of the protein, nucleic acid, and other components.

In some embodiments, the method comprises using both global electrodes and the edge electrodes to translocate a stranded molecule along the edge electrodes, measure electrical characteristics of a first segment of the polymer, then translocating the stranded molecule across the sensor edge; and measuring a second electrical signal through the edges when a second portion of the strand molecule is adhered to both edge portions of the at least one first electrode sheet and the at least one second electrode sheet, following the translocation of the stranded molecule and repeating until the entire length of the polymer is characterized. In some embodiments, the method comprises: linearizing the stranded molecule along the fluidic passage, e.g., by adjusting the magnitude and direction of the voltage across the fluidic passage. In some embodiments, the first portion of the stranded molecule is in a stretched conformation when the first portion of the stranded molecule is adhered to both edge portions of the at least one first electrode sheet and the at least one second electrode sheet of the at least one sensor.

In some embodiments, the sensor edge of the at least one sensor is chemically modified or functionalized with one or more chemical group(s) and the adhesion between the stranded molecule and the sensor edge of the at least one sensor occurs through electrostatic interaction, hydrogen bonding, hydrophobic interaction, intercalating interaction, ionic interaction, or a combination thereof between the stranded molecule and the chemical group(s).

In some embodiments, detecting the first electrical signal and/or the second electrical signal comprises applying a voltage between the at least one first electrode sheet and the at least one second electrode sheet and measuring a tunneling current between the at least one first electrode sheet and the at least one second electrode sheet and along the length of the stranded molecule attached to the sensor edge. In some embodiments, detecting the first electrical signal and/or the second electrical signal comprises applying a voltage across the global electrodes and measuring a voltage drop between the at least one first electrode sheet and the at least one second electrode sheet. In some embodiments, detecting the first electrical signal and/or the second electrical signal comprises measuring an ionic current flowing through the edge portions of the at least one first electrode sheet and the at least one second electrode sheet. In some embodiments, the method comprises: measuring an in-plane tunneling conductance across the first portion of the stranded molecule. In some embodiments, the stranded molecule is fully enclosed by the sensor edge.

In some embodiments, translocating the stranded molecule comprises adjusting a voltage applied to the edge sensors to dissociate the first portion of the stranded molecule from the edge portions of the at least one first electrode sheet and the at least one second electrode sheet, and adhering the second portion of the stranded molecule to the edge portions of the at least one first electrode sheet or the at least one second electrode sheet.

In some embodiments, the system comprises: one or more sensor devices disclosed herein; an electronic control system electrically connected to the one or more sensor devices, the electronic control system configured to apply an input electrical signal to the one or more sensor devices and to receive an electrical signal from the one or more sensor devices when a stranded molecule is adhered to the at least one sensor edge of the one or more sensor device; and a computer control system for analyzing the output electrical signal from the one or more devices to determine a characteristic of the stranded molecule.

Also disclosed are methods of making a sensor device. In some embodiments, the method comprises: depositing at least one first electrode sheet, at least one first insulating layer, and at least one second electrode sheet on a substrate; depositing one or more dielectric layer to the substrate and patterning the multiple deposited layers to form a lateral fluidic passage; applying a metal film, and, etching a vertical fluidic passage, by anodizing the metal film in an electrolyte solution resulting in a vertical fluidic passage in fluidic connection with the lateral fluidic passage and containing the edge sensors.

In some embodiments, the metal film comprises aluminum, magnesium, titanium, or a combination thereof. In a preferred embodiment, the metal film is an aluminum film. In some embodiments, the electrolyte solution is an oxalic acid. In some embodiments, the density and/or dimension of the plurality of nanochannels are controlled by adjusting the voltage and temperature of the anodization and/or the concentration of the electrolyte solution. In some embodiments, the method further comprises positioning a global electrode in each of the one or more microwells. In some embodiments, the aperture/vertical fluidic passage has a dimension of about 1 nm-5 nm.

The methods, sensors, and devices described herein have many advantages over the prior technologies. For example, the multi-modal simultaneous sequencing approach can sequence nucleic acids by directly recording the tunneling conductance of nucleic acid bases along the molecular axis via edge electrodes and across a single base or base pair of a nucleic acid molecule via in-plane electrodes. The methods, sensors and devices described herein can also sequence a nucleic acid molecule by recording the ionic conductivity of the buffer solution around the nucleic acid molecule constrained by the edge. The detection of both the tunneling conductance of a nucleic acid base and the ionic conductivity of the buffer solution around the nucleic acid base add specificity and accuracy of each subunit identification and can be coupled to optical or spectroscopic characterization of the stranded molecule subunit as it is attached to the electrodes.

Details of one or more implementations of the subject matter described in this specification are set forth in the accompanying drawings and the description below. Other features, aspects, and advantages will become apparent from the description, the drawings, and the claims. The multiple implementations and embodiments may be used singly or in combinations.

### BRIEF DESCRIPTION OF THE DRAWINGS

FIG. 1 is a schematic illustration of an exemplary edge read sensor components.

FIG. 2 depicts an edge sensor configuration.

FIG. 3A depicts a side view of an edge read sensor setup including edge sensor, global electrodes and optical characterization of a constrained polymer subunit.

FIG. 3B depicts a block diagram of electronics for an edge read sensor setup.

FIG. 4 depicts polymer motion control.

FIG. 5 illustrates tunneling conductance measurement between a pair of edge sensor electrodes that are chemically modified for characterization of a nucleic acid molecule.

FIG. 6 illustrates in-plane tunneling conductance measurement across a molecule, as opposed to along a molecule as shown in FIG. 5.

FIG. 7 depicts use of an edge sensor in a combined measurement of tunneling current along a polymer molecule and current flow through ionic solution surrounding a polymer molecule.

FIGS. 8A-8H illustrate an exemplary workflow for sequencing a nucleic acid molecule using the sensor device disclosed herein.

FIG. 9 depicts wafer-scale edge sensor fabrication.

FIG. 10 depicts multiple sensor device fabrication.

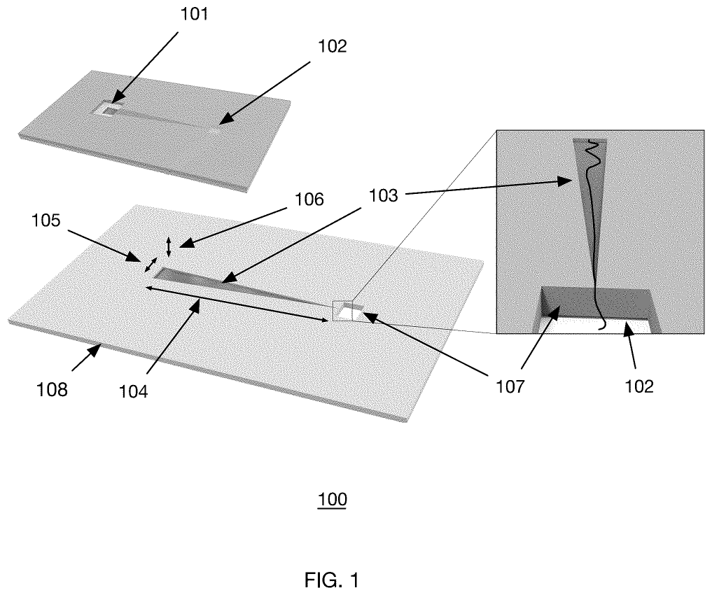

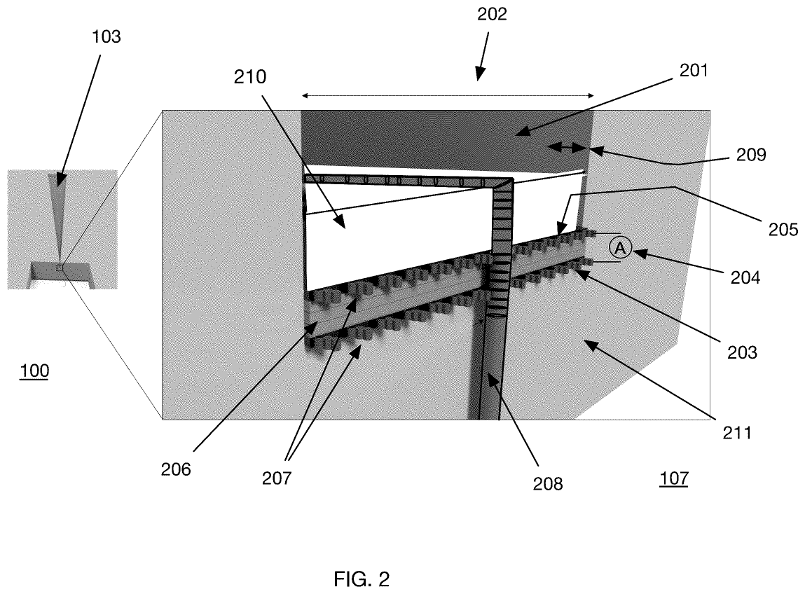

Throughout the drawings, reference numbers are re-used to indicate correspondence between referenced elements. Similar symbols identify similar components, unless context dictates otherwise. The drawings are provided to illustrate example embodiments described herein and individually are not intended to limit the scope of the disclosure. Examples illustrated in the individual Figures, can be arranged, substituted, combined, and, separated. The leading digit(s) of the elements reference numbers identify the figure where each element is first introduced. For example elements with reference numbers 100-107 are first shown in FIG. 1, elements with reference numbers 200-208 are first shown in FIG. 2, etc.

Unless defined otherwise, technical and scientific terms used herein have the same meaning as commonly understood by one of ordinary skill in the art to which the present disclosure belongs. The invented methods are applicable towards, but not limited to, characterization of multiple polymer types including biomolecules, such as deoxyribonucleic acid polymers (DNA), ribonucleic acid polymers (RNA), proteins and non-biological polymer types.

### DETAILED DESCRIPTION

### Edge Read Sensor Overview

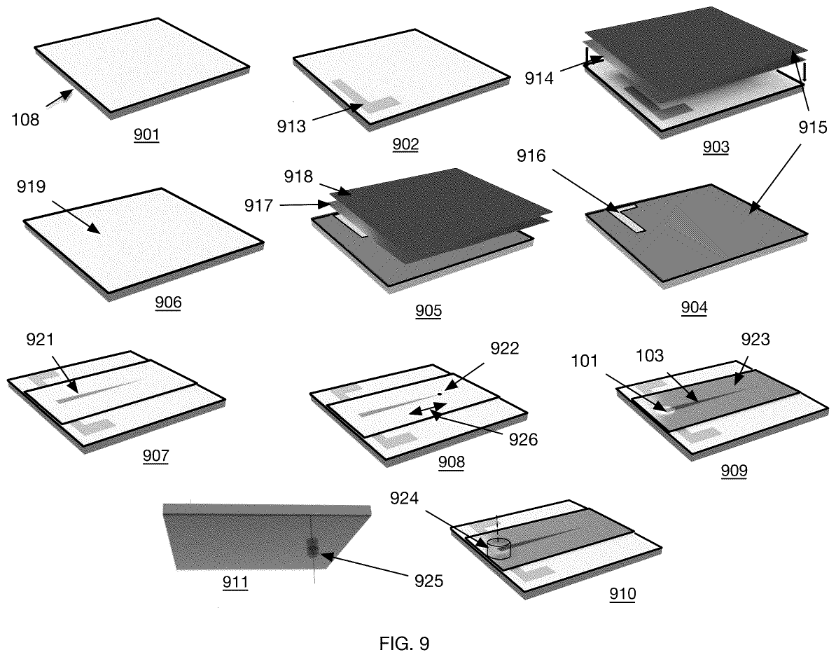

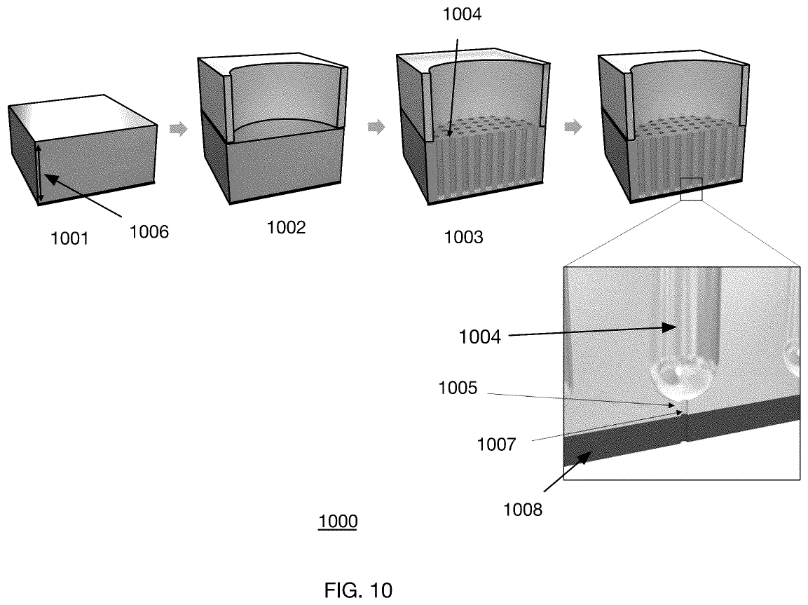

Referring now to FIGS. 1-3 , a general overview of an embodiment of the invented sensor device 100 for characterizing a polymer molecule. The sensor device 100 comprises: a lateral fluidic passage 103 disposed within a support structure 108 (e.g., a support structure comprising a semiconductor material). The lateral fluidic passage 103 connects to a vertical fluidic passage section 107 and the at least one sensor 203, 205 is disposed on the support structure with only the sensor edge exposed to the vertical fluidic passage section. The bottom 210 and walls 201 of the lateral fluidic passage and the walls 211 of the vertical fluidic passage, outside of the edge electrodes themselves are also dielectrics or the same material as the support structure 108 typically silicon. In some embodiments, the vertical fluidic passage and/or the sensor edge is in a closed-shape geometry 107, See FIG. 1, or a linear geometry forming one or more apertures 107. In another embodiment the devices involves a sensor edge comprising a plurality of conductor edges 203, 205, each exposed within a vertical fluidic passage and each individually electrically addressable. The vertical fluidic passage being a pore constructed as described in FIGS. 9 and 10 below.

A preferred embodiment, includes two or more global electrodes 301, 302 capable of providing a voltage to translocate the stranded molecule 208 through the lateral fluidic passage and the vertical fluidic passage. The stranded molecules movement is controlled by voltages applied to the global electrodes 301, 302 and the sensor edge 203, 205. The applied voltages cause the polymer to adhere to the sensor edge 203, 205 and a detector 305 is configured to detect a current between the at least one first electrode sheet 203 and the at least one second electrode sheet 205 as the stranded polymer molecule 208 is adhered to the sensor edge. The sensor device 100 can further comprise a loading well 101 and a fluidic reservoir 102 both in fluidic connection to the fluidic passage 103, in which the loading well 101 provides an inlet to the fluidic passage 103 and the fluidic reservoir 102 provides an outlet 201 from the fluidic passage. In an embodiment shown in FIG. 10 the loading well 1004, the sensor edge 1005 and the outlet 1007 are all vertically aligned. The support structure 108 can comprise a conductor material, semiconductor material, a dielectric material, glass, plastic, polymeric material, ceramic material, or others commonly used in fabrication of fluidic devices. In some embodiments, the support structure comprises a semiconductor material. The term “semiconductor material” refers to any material common in semiconductor industry. Examples of semiconductor materials that can be employed as the substrate 108 include, but are not limited to, Si, SiGe, SiGeC, SiC, Ge alloys.

In some embodiments, the lateral fluidic passage section is about 50-500 nm in width 105, 1-15 mm in length 104, and/or 1-100 nm in height 106. In some embodiments, the lateral fluidic passage section has an aspect ratio of height to length in a range from about 1:10 to about 1:1000, e.g., from about 1:50 to about 1:500. In some embodiments, the lateral fluidic passage section has a cross-sectional area of about tens to hundreds of nm². In some embodiments, the lateral fluidic passage section and/or the vertical fluidic passage section has a dimension configured to accommodate no more than one stranded polymer molecule 208.

In some embodiments, the tapered horizontal channel 103 has a channel width 105 of 100 nm at its starting point (near the loading well 101) and length 104 of 10 micrometers with an aspect ratio of 1:100 and has an acute angle of about 0.57° at the end 201 where the horizontal channel 103 and the vertical channel 107 intersect. At this aspect ratio, patterning a via through the channel that crosscuts the wedge 100 nm from the “close point” end 201 generates an edge width 202 of 1 nm. The edged width is determined by an offset 209 of the via relative to the open end of the lateral fluidic passage. An offset of 200 nm towards the open end of the wedge generates the edge width of 2 nm. For a 10 nm edge width 202, using a wedge shaped channel 103 with dimensions as described immediately above, the via 107 must be offset 209 by 1 micron. The width 202 of the edge electrode is determined by the location of the vertical channel 107 along the long axis 104 of the lateral fluidic channel. In some embodiments, the lateral fluidic passage section 103 and/or the vertical fluidic passage section 107 has a chemically modified inner surface. In some embodiments, the inner surface is chemically modified to carry a charge. In some embodiments, the inner surface comprises glass, a self-assembled monolayer, an organic molecule, aluminum oxide, a lipid bilayer, or a combination thereof. In some embodiments, the lateral 103 and/or vertical 107 fluidic passage section is filled with hydrogel. In some embodiments, the hydrogel is naturally occurring or synthetic. In some embodiments, the hydrogel is agarose, polyacrylamide, PVA, or a combination thereof. In some embodiments, the lateral and/or vertical fluidic passage section comprises nanoparticle matrices.

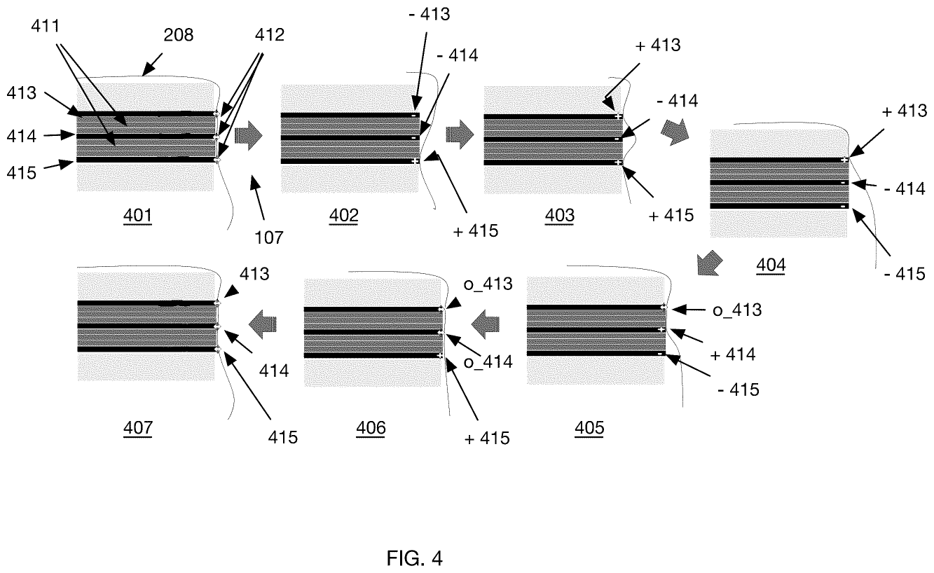

The invention includes sensors, devices, and related methods for characterizing a biomolecule by monitoring an electrical signal (e.g., electric tunneling current and/or ionic conductivity) along a portion of a biomolecule 208 as the biomolecule translocates, as described in FIG. 4 below, along an exposed edge portion 207 of the sensor. The device takes advantage of the atomic thinness of the electrodes 203, 205. In some embodiments, the thickness of each electrode is equal to, for example, a single nucleic acid base pair length (e.g., 0.34 nm) for a total thickness of about 1.6 nm. For example, two graphene electrodes 203, 205 in the device can be separated by the controlled number of dielectric layers 206 (e.g., hBN layers) forming an open circuit without nucleic acid present. When a molecule 208 (e.g., a nucleic acid molecule) is in contact with the edge 207, the graphene edge, being a conductor, closes the circuit and allows current to run through the longitudinal length of the polymer. The current depends on the characteristics of the molecule (e.g., base pair sequence) completing the circuit between the first edge electrode 203 and the second edge electrode 205. The edge of the electrodes 207, in a preferred embodiment are made of graphene, and the dielectric layers 206, e.g., the hBN layers can be functionalized with moieties forming hydrogen/ionic/pi-stacking bonds with the molecule 208 such that the polymer being investigated remains in contact with the edge 207. The graphene edge moieties can be conjugated and provide direct charge transfer from the graphene electrode into the molecule while also providing a prescribed number of degrees of freedom for the translocation of the molecule. The edge of the insulating layers (e.g., hBN layers) 206 can also be functionalized with aliphatic moieties that restrict electron tunneling while supporting the prescribed molecule retention. The energetics of the molecule (e.g., nucleic acid molecule) retention is such that it allows axial translocation of the molecule during electrophoresis. Such architecture can ensure that the variable resistance arm of the electrical circuit (i.e., the molecule in contact with the edge) is composed of only one to a few monomers (e.g., 1-3 base pairs) ensuring high accuracy. The number of monomers can be predetermined by the spacing 204 between the electrode sheets 203, 205. The wedge-shaped geometry and the dimensions 104, 105, 106 of the lateral channel 103 and width 202 of the edge electrodes 203, 205 of the device ensure only a single polymer molecule 208 (e.g., a single nucleic acid molecule) is read at any given time.

In some embodiments the edge sensor further includes optical or spectroscopic characterization of the segment of the polymer 208 while attached to the edge sensor. A light source is focused on the edge sensor with a detector detecting light reflected from the edge sensor 203, 205. This optical characterization of each polymer segment as it traverses the edge sensor further enhances specificity and accuracy of subunit and therefore sequence determination.

The closely positioned electrode array at the edge, supplemented by the global electrophoresis electrodes 301, 302 in the buffer volume, can assist in controlling the polymer 208 translocation speed and direction, with single subunit, or, in the case of nucleic acid a single base-pair resolution, by a proscribed alternating current modulation of the electrode array, see FIG. 4 and accompanying discussion below. The edge geometric parameters (e.g., ˜10 nm long) 204 and the overall construction as described herein, overcome the lithographic limitations of creating sub 3 nm nanopores and can ensure at-scale fabrication using current semiconductor manufacturing practices. The electronic control of nucleic acid translocation by the edge (FIG. 4) can be performed by a power supply 304 applying voltages to the global 301, 302 and edge 203, 205 electrodes at ˜KHz to MHz frequencies thus allowing sequencing at microseconds per base pair (μs/bp) rates. Translocation direction can also be controlled electronically via such modulation thus allowing to “re-wind” and re-sequence DNA as desired.

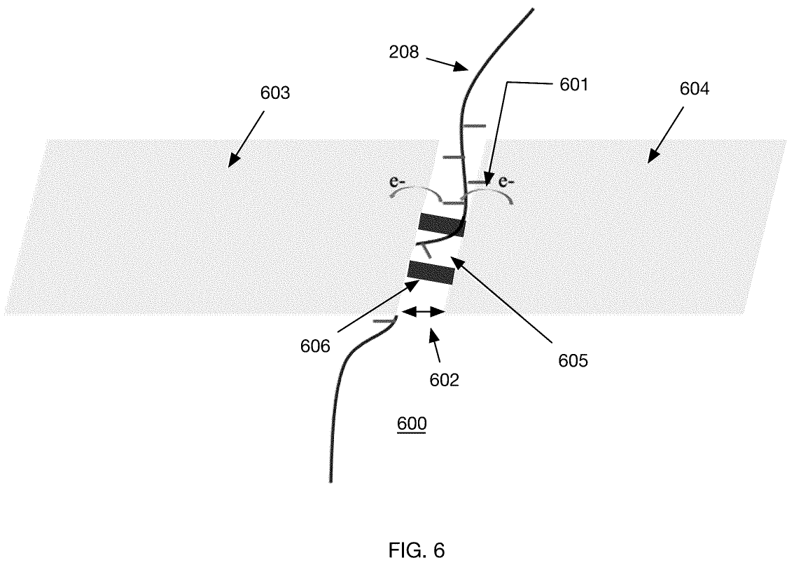

The geometry of the sensor edge can be linear with “sandwich” structure only on one side of the channel that DNA flows through as shown in FIG. 2 or the geometry can be closed-loop 600, as shown in FIG. 6, where the edge of the electrode 603 encloses a molecule 208 on all sides irrespective of the perimeter geometry. FIG. 6 shows a molecule passing through a pore 604 with electrode material the target molecule 208 to be characterized is fully surrounded (e.g., with a closed-loop edge) by the sensor edge. The sensor edge 604 of a molecule sensor can, in a horizontal cross-sectional view, in which the sensor edge encloses a molecular of interest, have a circular geometry, an ellipse geometry, a triangle geometry, or, square, as shown in FIG. 6, or rectangular geometries.

In preferred embodiments, the at least one first electrode sheet 203 and/or the at least one second electrode sheet 205 comprises a two-dimensional electrically conductive material. In some embodiments, the two-dimensional electrically conductive material are selected from: gold, titanium nitride (TiN), poly(3,4ethylenedioxythiophene) polystyrene sulfonate (PEDOT:PSS), molybdenum disulfide (MoS2), copper, platinum, nickel, or, graphene, or a combination thereof. In some embodiments, the at least one first electrode sheet 203 and the at least one second electrode sheet 205 comprise a same electrically conductive material or different electrically conductive materials. In some embodiments, the at least one first electrode sheet 203, the at least one second electrode sheet 205, and the at least one first insulating layer 206 have adjacent exposed edge portions within the vertical fluidic passage which collectively form the sensor edge. In some embodiments, the edge electrode comprises one, two, three, four, or more sheets of electrodes each individually electrically addressable. In preferred embodiments, electrode sheets comprise graphene. In some embodiments, some of the electrode sheets comprise graphene and other electrode sheets do not comprise graphene.

In some embodiments, the at least one first insulating layer 206 comprises one, two, three, four, or five or more first insulating layers. In some embodiments, the at least one insulting layer comprises ceramics, diamond, two-dimensional polymers, self-assembled monolayers or bilayers, and/or other electrically insulating organic and inorganic moieties. In some embodiments, the ceramics comprises Si3N4, Al2O3, HfO2, or a combination thereof. In some embodiments, the at least one first insulating layer 206 comprises one or more atomically thin sheets. In some embodiments, the at least one first insulating layer 206 comprises boron nitride. In some embodiments, the at least one first insulating layer 206 comprises hexagonal boron nitride (hBN). In some embodiments, the at least one first insulating layer comprises three sheets of boron nitride or hBN. In some embodiments, the at least one first electrode sheet 203 and the at least one second electrode sheet 205 each comprises a single graphene sheet, and the at least one first insulating layer 206 comprises three sheets of hBN as shown in FIG. 2.

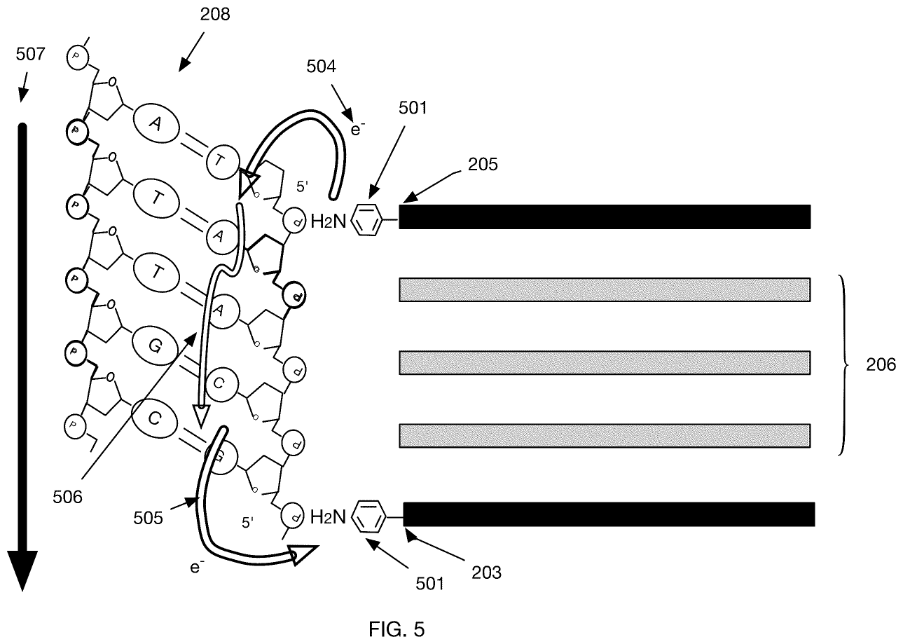

In some embodiments, the sensor edge is configured to partially or fully surround the stranded molecule 208. In some embodiments, the sensor edge is in a closed-loop geometry or a linear geometry. In some embodiments, the sensor edge is in an open linear geometry and has a width 202 of about 5-10 nm. In some embodiments, the sensor edge is in a closed-loop geometry having a cross-sectional dimension 204 of about 2-3 nm. As shown in FIG. 5, in some embodiments, the sensor edge 203, 205 is functionalized with one or more chemical group(s) 501. In some embodiments, the functionalization comprises hydrogenation, hydroxylation, carboxylation, carbonylation, amination, or a combination thereof. In some embodiments, the sensor edge is configured to minimize the tunneling electrical resistance between the sensor edge and the stranded molecule, e.g., configured with a charge-injecting group. In some embodiments, the sensor edge is configured to enhance the tunneling electrical interaction between the sensor edge and the stranded molecule. In some embodiments, the configuration enhances binding with the stranded molecule through hydrogen bond interaction, electrostatic interaction, hydrophobic/intercalating interaction, biomolecular binding, or a combination thereof.

In some embodiments, the sensor edge comprises a functional group selected from the group consisting of: carbonyl, carboxyl, hydroxyl, aldehyde, carboxylate, ester, or amine functional group, or a combination thereof. In some embodiments, the sensor edge is functionalized with a p-phenylenediamine moiety 501. In some embodiments, the edge portions of the at least one first electrode sheet, the at least one second electrode sheet, and the at least one first insulating layer 206 are functionalized with a same functional group or different functional groups. In some embodiments, a voltage drop between the at least one first electrode sheet 203 and the at least one second electrode sheet 205 is capable of being measured when the stranded molecule is adhered to the sensor edge and a voltage is applied across the at least one first electrode sheet and the at least one second electrode sheet. As shown in FIG. 4, in some embodiments, the sensor comprises: at least one second insulating layer located on a surface of the at least one first electrode sheet 408 or the at least one second electrode sheet 410, and at least one third electrode sheet 409 located on a surface of the at least one second insulating layers. In some embodiments, the at least one first electrode sheet and the at least one second electrode sheet comprise graphene and the at least one third electrode sheet does not comprise graphene.

Disclosed herein includes an electronic molecule sensor 100 comprising alternating electrode sheets 203, 205 separated by alternating insulating layers 206. The sensor typically comprises an electrode-dielectric stack including a plurality of layers/sheets with at least one insulating layer disposed between each of the electrode sheets. The electrode sheet can be atomically thin and electrically conducting. In some embodiments, the molecule sensor comprises a sandwich structure with graphene on the top 203 and bottom 205 and insulating sheets 206 between the at least two graphene sheets. The insulating sheets can also be atomically thin sheets. In some embodiments, the insulating sheets can be boron nitride or hexagonal boron nitride (hBN). The electrodes and the insulating layers can both comprise one, two, three (see FIGS. 4, 413, 414, 415), or more sheets. In some embodiments, the layers are in sheet form, which may comprise a single sheet or multiple sheets of 2-D conductive or dielectric material. The molecule sensor can comprise an edge exposed to a solution with analyte (e.g., a target molecule 208). The edge 207 used herein refers to a lateral or an end portion of the electrode sheets 203, 205 and the insulating layer 206. A target molecule 208 to be characterized can be partially surrounded or fully enclosed by the edge 207. The length 202 of the exposed edge can be on the nanometer scale. Referring to FIG. 5, the edges of the electrode sheets (e.g., graphene sheets), the insulating sheets, or both, can be chemically modified by etching the electrodes and/or the insulating layers at an edge portion 207, FIG. 2 and/or introducing a functional group 501 at the etched edge. The edges of the electrode sheets and the insulating sheets can be modified with a same chemical group or different chemical groups, or a same biological group or different biological group (e.g., a single stranded DNA binding protein or domain). Voltage (e.g., DC current, AC current, other waveforms, or electrical impedance) can be applied across the two electrode layers 203, 205 (e.g., graphene sheets) such that there is a voltage drop across the exposed electrode (e.g., graphene) edges when a target molecule is adhered to the sensor edge. The presence and identity of an analyte can be detected by a change in tunneling current 504, 505, 506 caused by the position of the analyte with respect to the electrode edges.

The edge read can be designed to directly measure the conductance 506 of a target biomolecule 208 under investigation to elucidate its primary structure-subunit/monomer composition/sequence. At the core of the technology is the ability to inject, extract, and measure electrical current across a small and well-defined portion of the investigated molecule. For example, in the context of nucleic acid sequencing, each different base residing near an electrode layer can lead to different tunneling current across two closely spaced electrodes 203, 205 (e.g., graphene electrodes) because of the different electronic level structure of the bases. The edge sensor can therefore characterize each nucleotide base by measuring the tunneling current across the two closely spaced electrodes as the nucleic acid translates 507 across the electrode edges. The sensor can be used to characterize any stranded molecule or biomolecule 208 composed of multiple units and monomers. In some embodiments, a target molecule can be a biomolecule, including for example polymers, polynucleotides, oligonucleotides, polypeptides, polysaccharides, or combinations thereof.

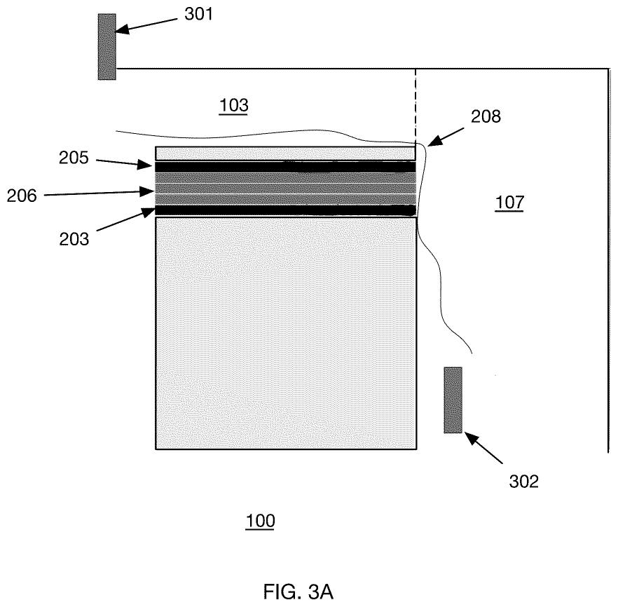

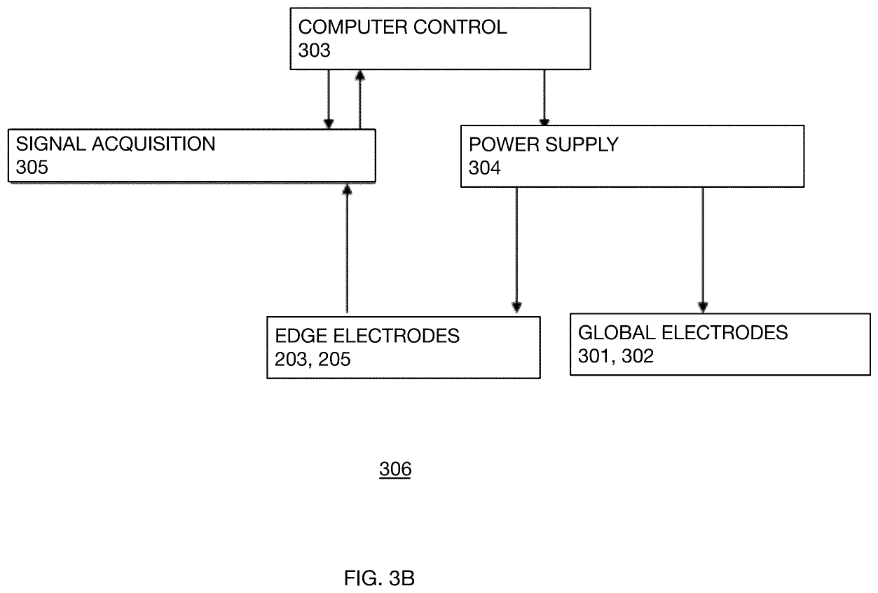

Referring to FIGS. 3A and 3B, the systems for characterizing a stranded molecule comprises: one or more sensor devices 100; an electronic control system 306 electrically connected to the one or more sensor devices, the electronic control system configured through computer control 303 to apply, through a power supply 304, an input electrical signal to the one or more sensor devices including the edge electrodes 203, 205 and the global electrodes 301, 302 and to receive an electrical signal through signal acquisition devices 305 from the one or more edge electrodes within each of the sensor devices 100 when a stranded molecule 208 is adhered to the at least one sensor edge 203, 205 of the one or more sensor device 100; and a computer control system 303 for acquiring 305 analyzing the output electrical signal from the one or more devices to determine a characteristic of the stranded molecule 208.

In some embodiments, the electrode sheets comprise one or more graphene sheets. A graphene sheet has two types of electro transfer sites—edge and basal. The graphene edge plane atoms 207 have been reported to possess significantly higher electron transfer rates and reactivity compared to basal planes in electrochemical studies. There are several benefits of employing graphene as opposed to any other conductive thin-layer material. First, the graphene sheet has a single-atom thickness (3.4 Å), which defines the molecule/electrode interaction degrees of freedom (DOF) parameter field in the vertical dimension. Additionally, this thickness corresponds to the single nucleic acid base-pair planar dimension on the sub-Angstrom level, which adds to the enhanced measurement resolution. Second, graphene has high current-carrying capacity overall and high current injection density at the edge in particular. Third, the edge of the graphene has the ability to be functionalized with desired chemical functionality as shown in FIG. 5. The thinness of the edge design, facilitated by the mono-layer fabrication, allows interrogation of a single or a small number of polymer units (e.g., bases, amino acids). This can minimize the deconvolution required to accurately determine the polymer sequence.

The non-biologic nature of the edge sensor materials and the disclosed approaches allows greater flexibility in the aspect ratios of the device unconstrained by the structure of protein and derivative components as in prior art sequencing procedures. This enables flexibility in the device to allow the direct characterization of polymers, such as sequencing RNA without prior reverse transcription into DNA. This approach also enables the direct characterization/sequencing of proteins and other polymers without derivatization or other modifications of the polymers to be characterized. Additionally, because biological molecules are sensitive to post-synthesis modification, a non-biological approach as used herein facilitates the characterization of both modified and unmodified molecules and their discrimination. For example, the sequencing of methylated DNA through a thin-layer electronic edge sensor provided herein can allow the detection of methylated DNA bases by detecting changes in the electric current flowing through the base under detection. Similarly, non-limiting post-translational modification to proteins, such as phosphorylation, glycosylation, SUMOylation, ubiquitination, etc. can be detected.

Referring to FIG. 6, in some embodiments, the sensor edge forms a close-loop shape. The closed-loop is an alternate edge geometry that allows for the tunneling current measurement both along the length of a segment of the polymer as shown in FIG. 5 and across the polymer 601 as shown in FIG. 6. With the critical dimension of this geometry being the loop diameter 602, the edge length of any closed-loop geometry is at least 3.14 (π) times greater. Accordingly, in some embodiments, the diameter dimension 602 of a close-loop edge is about 2 nm to 3 nm. In some embodiments, the dimension of a closed-loop edge can be greater than 3 nm to accommodate a larger molecule to be characterized.

The insulating layer 206 is composed of non-conducting dielectric material. The dielectric material in the insulating layer can possess one or more of the following characteristics: (1) being thickness-definable on the sub-nanometer atomic level; (2) capable of maintaining sufficient dielectric properties at a defined thickness; and (3) in a preferred embodiment capable of being synthesized on graphene. In some embodiments, the insulating layer comprises boron nitride, such as hexagonal boron-nitride (hBN). Similar to graphene, a single hBN layer has a thickness of about 3.4 Å, with a crystallographic match and high energy of adhesion to graphene. Other dielectric materials that can be used in the insulating layer include, but are not limited to, ceramics (Si3N4, Al2O3, HfO2, ZrO2, HfSiO4, ZrSiO4, or any combination thereof), diamond-like carbon, carbon boron nitride, 2D polymers, self-assembled monolayers or bilayers and other electrically insulating organic and inorganic moieties. The appropriate dielectric material or a combination thereof can be selected for the sandwich structure based on layer thickness, dielectric properties, fabrication appropriateness, as well as chemical properties with respect to edge functionalization.

The at least one first insulating layer can comprise one or multiple insulating layers. The multiple insulating layers, such as three layers 206 shown in FIG. 2, can comprise a same dielectric material or different dielectric materials. In some embodiments, at least one first insulating layer comprises boron nitride, optionally the at least one first insulating layer comprises hexagonal boron nitride (hBN). For example, as shown in FIG. 2, the insulating layer 206 can comprise three sheets of boron nitride or hBN. The thickness of the insulating layer(s) can vary from single-atom thick (e.g., one hBN sheet) to hundreds of microns and even millimeter (e.g., silicon substrate and polymers, respectively). In some embodiments, it may be necessary to functionalize graphene electrodes with a certain moiety while leaving hBN edge bare as shown in FIG. 5, or have different functional groups attached to electrode and dielectric edges.

In some embodiments, as shown in FIG. 3, the sensor device comprises a fluidic passage 103 disposed within a support structure 108, two or more global electrodes 301, 302 capable of providing a voltage to translocate the stranded molecule 208 through the lateral fluidic passage 103 vertical fluidic passage 107, at least one edge sensor 203, 205, 206 described above disposed in the support structure with the sensor edge exposed to the fluidic passage 107, and a detector 305 configured to detect a current between the at least one first electrode sheet 203 and the at least one second electrode sheet 205 as the stranded molecule 208 is adhered to the sensor edge. The adhesion of the stranded molecule to the sensor edge can occur through physical and/or chemical interactions between the stranded molecule and the sensor edge such as the functional groups 501 of the sensor edge.

The sensor device can comprise 1 to 10 or more sensors as described above. In some embodiments, the number of sensors in a sensor device can be 1 to millions of sensors either each with a distinct fluidic passage 103 or multiple edge sensors 203, 205, 206 sharing a single fluidic passage.

In some embodiments, the sensor device described herein is configured to allow a single stranded molecule 208 to flow through the fluidic passage at any given time. Several strategies can be used to achieve this goal, including, for example, geometric constrain, intermolecular repulsion/hindrance, and/or surface charge constraints. For example, a micro/nanoscopic channel 103 with high-aspect-ratio leading to the edge portion 107 can ensure multi-strand molecule exclusion. The small cross-sectional area of the channel 107 (e.g., tens to hundreds of nm2) coupled with its high aspect ratio can make entry of more than one nucleic acid strand into the channel electrostatically and entropically unfavorable. In some instances, the geometry of the edge read channel 103 bears a wedge shape of high aspect ratio with its acute angle bearing a 5-10 nm wide opening, 202. Intermolecular repulsion can also be used to hinder the entry of more than one stranded molecule into the channel. For example, in solutions with low osmolarity, the negatively charged nucleic acid molecules take linear configurations. Within a nanoscopic channel 201, such linear charge distribution ensures electrostatic DNA/DNA repulsion and thus exclusion of all but one DNA molecule from the channel. In some embodiments, the interior surface of the channel can be composed of or chemically modified to compose of materials bearing negative charge (e.g., glass, or self-assembled monolayer with exposed phosphate group) to further promote exclusion of negatively charged molecules from the channel to the degree necessary to ensure single molecule interrogation requirement. For example, in the case of nucleic acid sequencing, the interior surface of the channel can be chemically modified with negatively charged molecules or chemical groups to ensure a single nucleic acid strand in the channel at any given time. Similarly, the interior surface of the channel can also be chemically modified with positively charged molecules or chemical groups to promote exclusion of positively charged molecules. FIG. 5 shows chemically modified 501 edge electrodes 203, 205.

The global electrodes 301, 302, also referred to as buffer-biasing electrodes, can provide an electrical voltage bias across the fluidic passage 103, 107. The global electrodes can control the global electrophoretic force applied on the stranded molecule 208 by applying a prescribed bias across the entire liquid volume in the device. A voltage across the global electrodes 301, 302 can be adjusted to apply a force to the translated molecule such that the translation direction and rate through the fluidic passage can be controlled as desired. The voltage can be adjusted to produce an electrophoretic force along the lateral fluidic passage 103 to facilitate the translocation of a stranded molecule from the loading well 101 to the opening near the top of the vertical edge. Once the molecule enters the vertical channel 107, the voltage can be adjusted to produce an electrophoretic force along the vertical channel. The edge electrodes 203, 205 (e.g., the graphene electrodes) can also be used to manipulate molecular movement on macro-and nanoscale. When the target molecule is in physical contact with the sensor edge 203, 205, the tunneling conductance 506 between the electrodes can be measured. The graphene electrodes can therefore induce controlled molecular motion at the nanoscale in the vicinity of the edge as shown in at least FIGS. 4 and 5.

In a non-limiting exemplary embodiment, a sensor device can comprise a fluidic passage 103, 107 disposed in a support structure 108, wherein the fluidic passage comprises a lateral fluidic passage 103 and a vertical fluidic passage 107, wherein the lateral fluidic passage is in fluidic connection with the vertical fluidic passage, optionally the support structure comprises a semiconductor material, two or more global electrodes 301, 302 capable of providing a voltage to translocate the stranded molecule through the lateral 103 and vertical 107 fluidic passages, at least one sensor disposed in a bottom of the lateral fluidic passage 103, wherein the sensor comprises at least one first graphene sheet 203, at least one second graphene sheet 205, and at least one first insulating layer 206 disposed between the at least one first graphene sheet and the at least one second graphene sheet, and wherein the sensor comprises a sensor edge 207 capable of interacting with the stranded molecule 208, thereby modulating a tunneling current between the at least one first graphene sheet 203 and the at least one second graphene sheet 205, and a detector 305 configured by a computer controller 303 to detect an electrical signal between the at least one first graphene sheet and the at least one second graphene sheet when the stranded molecule is adhered to the sensor edge.

In some embodiments, detecting the first electrical signal and/or the second electrical signal comprises applying a voltage between the at least one first electrode sheet 203 and the at least one second electrode sheet 205 and measuring a tunneling current between the at least one first electrode sheet and the at least one second electrode sheet. In some embodiments, detecting the first electrical signal and/or the second electrical signal comprises applying a voltage across the global electrodes 301, 302 and measuring a voltage drop between the at least one first electrode sheet 203 and the at least one second electrode sheet 205. In some embodiments, detecting the first electrical signal and/or the second electrical signal comprises measuring an ionic current flowing through the edge portions of the at least one first electrode sheet and the at least one second electrode sheet. In some embodiments, the method comprises: measuring an in-plane tunneling conductance 601 across the first portion of the stranded molecule, applying voltages across the global electrodes and/or the first electrode sheet and the second electrode sheet to incrementally move the molecule 208 along the lateral and vertical passages and then measuring the second electrical signal. In some embodiments, the stranded molecule is fully enclosed by the sensor edge.

In some embodiments, as shown in FIG. 4, translocating the stranded molecule comprises: dissociating 402 the first portion of the stranded molecule from the edge portions of the at least one first electrode sheet and the at least one second electrode sheet, and adhering 403 the second portion of the stranded molecule to the edge portions of the at least one first electrode sheet or the at least one second electrode sheet. In some embodiments, dissociating the first portion of the stranded molecule from the edge portions comprises dissociating 402 the first portion of the stranded molecule from the edge portion of the at least one first electrode, followed by dissociating 404 the first portion of the stranded molecule from the edge portion of the at least one second electrode. In some embodiments, the stranded molecule is in a relaxed state during said dissociating. In some embodiments, the stranded molecule remains adhered to at least one edge portion of the at least one first electrode sheet or the at least one second electrode sheet 401-404. In some embodiments, dissociating the first portion of the stranded molecule and adhering the second portion of the stranded molecule comprises (1) adjusting the magnitude and sign of the voltage between global electrodes 301, 302 and the edge electrodes 412 and/or (2) adjusting the interaction between the edge electrodes and the stranded molecule chemically by derivatizing the edge electrodes 203, 205 or the dielectric layers 206. In some embodiments, translocating the stranded molecule moves the stranded molecule across the sensor edge by a portion or a unit of the stranded molecule. In some embodiments, the stranded molecule is partially or fully surrounded by the sensor edge.

In some embodiments, the characteristic of the first portion of the stranded molecule comprises polynucleotide sequence, polynucleotide methylation, polypeptide sequence, protein glycosylation, protein-polynucleotide binding event, or a combination thereof. In some embodiments, the stranded molecule is a nucleic acid molecule and determining the characteristic of the first portion of the stranded molecule comprises determining a sequence information of a nucleic acid base. In some embodiments, the method comprises: depositing a sample comprising the stranded molecule in a loading well 101 of the sensor device 100. In some embodiments, the stranded molecule comprises a polymer, a polynucleotide, an oligonucleotide, a polysaccharide, a polypeptide, or a combination thereof.

The sensor 100 can comprise three electrode sheets 413, 414, 415 each separated by insulating layers 411 as shown in FIG. 4. In some embodiments, increasing the number of insulating layers (e.g., hBN layers) 206 and/or electrode layers 203, 205 can mitigate potential defects leading to current leakage or dielectric breakdown. For example, as shown in FIG. 4, the sensor can further comprise at least one insulating layer located on a surface of the at least one first electrode sheet 415 and a second insulating layer located above the at least one second electrode sheet 414, and at least one third electrode sheet 413 located on a surface of the second insulating layers. The second insulating layer can contain a dielectric material that is the same as or different from the first insulating layer. The at least one third electrode sheet can contain an electric material same as or different from the first electrode sheet and/or the second electrode sheet. In some embodiments, the at least one first electrode sheet and the at least one second electrode sheet comprise graphene and the at least one third electrode sheet does not comprise graphene.

In some embodiments, the molecule sensor can comprise multiple (e.g., one, two, three, four, five or more) individually addressable graphene electrodes separated by dielectric layers. FIG. 4 shows three individually addressable electrodes. Such arrangement in the “read-mode” can mean double-reading a segment of molecule (e.g., a segment of a nucleic acid sequence) on one pass as well as providing enhanced translocation control as shown in the sequence 401-407 of FIG. 4.

The electrical control system 306 affects the edge electrode and target molecule 208 interaction. For example, such a moiety can possess a certain length/rotational angle such that it is free (DNA-interacting) end motion with respect to the DNA axis, would be co-planar with no electrical charge on the edge electrodes 412, step 401 asl well as have the absolute value around 3.4 Å—the inter-base distance. In such an arrangement, the nucleic acid 208 can be translocated across the edge one base-pair per electrical modulation cycle as shown in items 413-415 where the leading +or-indicates the electrical charge applied to the graphene edge electrodes for modulation steps 402-406 resulting in step 407 where a second segment of the molecule of interest 208 adheres to the edge electrodes with a charge related to measurement 413, 414, 415 applied. The control system can apply a first voltage to the edge electrodes 413-415 that is related to measurement of the electrical characteristics of the polymer 208 as shown in the first and last steps 401, 407 and a second voltage +/− 413-415 for translocation of the polymer 208 as shown in translocation steps 402-406.

In some embodiments, the sensor edge is in a linear, open, geometry as shown in FIG. 3A or in a closed, nanopore 107 geometry. A linear edge can decrease the critical dimension that needs to be lithographed and improve manufacturability compared to a closed nanopore 107 due to the current nanotechnology bottleneck. For example, by contrast in FIG. 6 and the enclosed geometry of FIGS. 1, 107, a nanopore 3 nm in diameter 602 is more challenging to pattern at scale and with precision than a linear edge feature 204 of 10 nm using current production lithography tools, even though the edge length for both geometries is roughly the same: ˜10 nm. This consideration lifts one of the most challenging aspects for adopting solid-state nanopore technology—sub 5 nm planar dimensional control. A linear edge can also allow for enhanced electrostatic molecule translocation control. In an enclosed geometry 107 such as a nanopore, the electrostatic field flux equals zero anywhere within the perimeter of the electrode loop. In some embodiments, that means that the molecule (e.g., DNA) is experiencing zero net electrostatic force within the plane and inside the enclosed electrode loop, which would potentially result in it freely oscillating within the pore. When the geometry of the edge electrode, however, is linear, and, open or not fully enclosed, the electric field flux cross product is a non-zero vector which results in either an attractive or repulsive force on the negatively charged DNA molecule depending on the electrode bias sign see + or − indices of FIG. 4 such as + or − charges preceding the edge labels 413 through 418 or neutral such as no electrical charge, as indicated for example with the label 412 of step 401 and step 407.

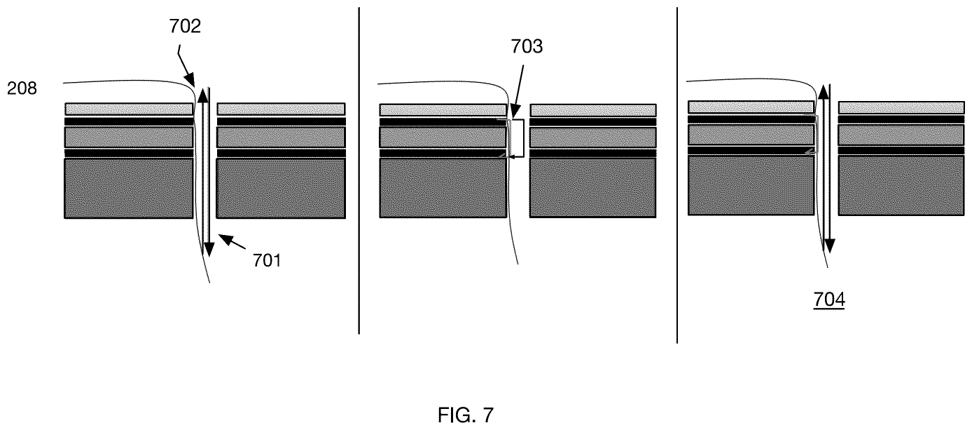

Referring to FIG. 7, characterizing a polymer 208 includes measuring the ionic flow 701, 702 around a polymer molecule 208 within a pore 107 of the sensor device giving an assessment of the excluded volume of ions within the pore with the polymer molecule present, measuring the tunneling current 703 along the length of a segment of the molecule within the pore, or simultaneously making both measurements 704.

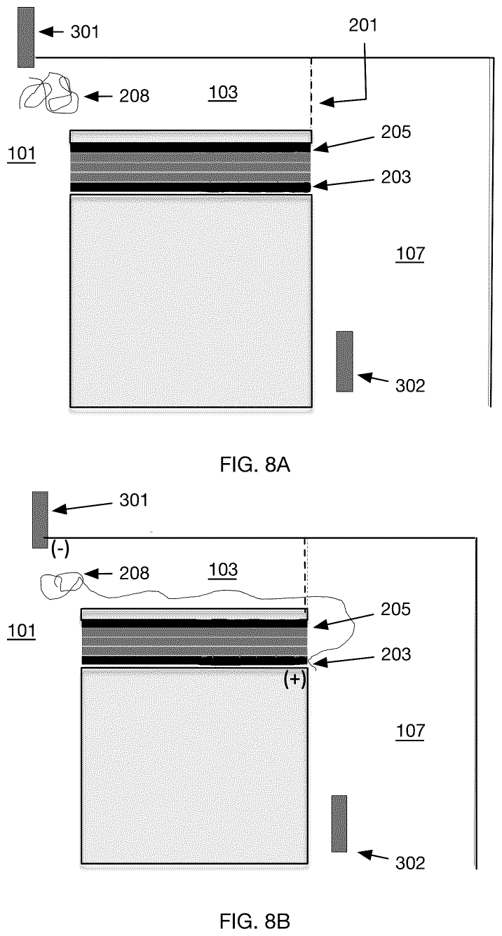

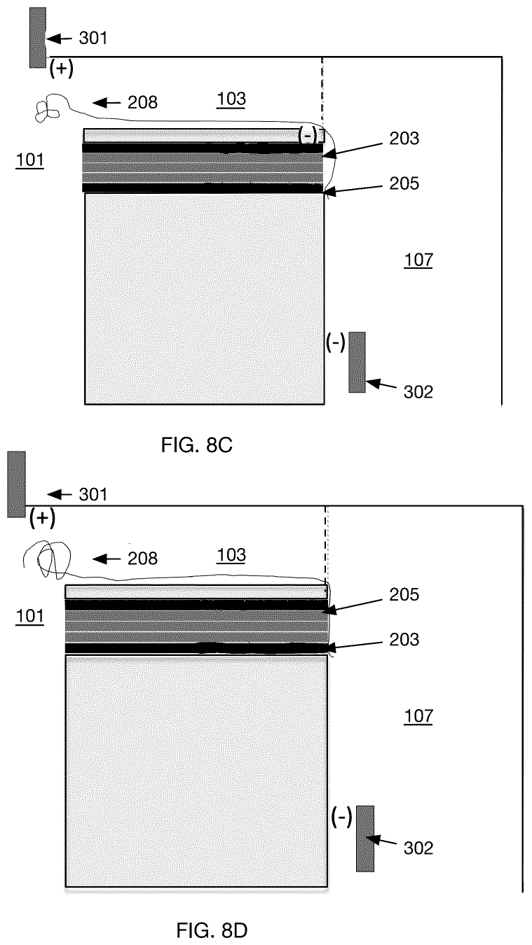

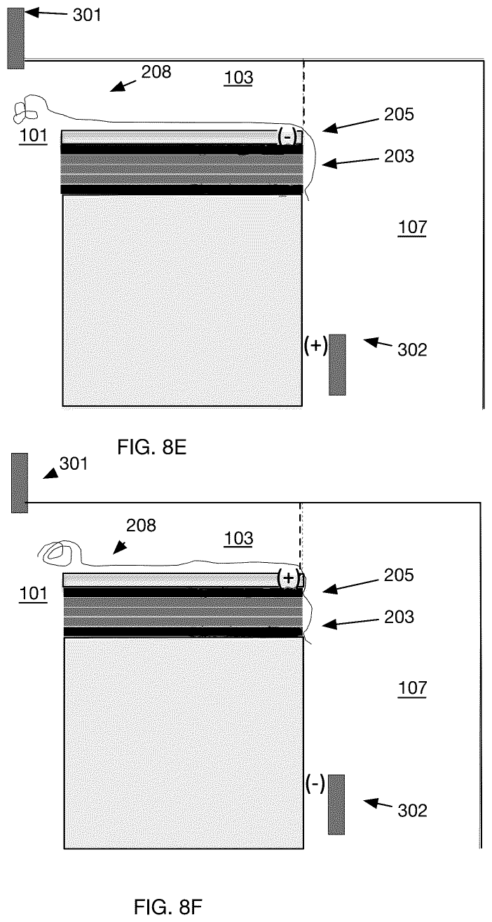

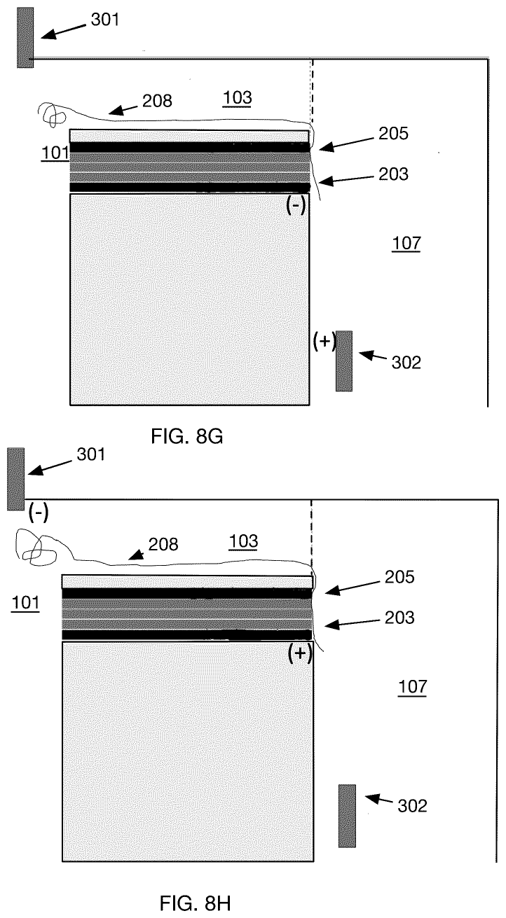

Disclosed herein include methods of characterizing a stranded molecule. As shown in FIGS. 8A through 8H, in some embodiments, the method comprises: providing a sensor device disclosed herein FIG. 8A; applying a voltage across the global electrodes 301, 302 and/or also across one or more of the electrode sheets therefore across the fluidic passage 103, 107 of FIG. 8B to produce a current flow through the fluidic passage 103, 107, causing a single stranded molecule 208 to translocate through the fluidic passage one segment at a time FIG. 8B and allowing said single stranded molecule to interact with the sensor edge of the at least one sensor 205, note in the example of FIG. 8C shown, sensor edge 203 is negatively charged such that a negatively charged molecule 208 is repulsed at 203 but adheres at 205; detecting a first electrical signal between the at least one first electrode sheet 203 and the at least one second electrode sheet 205 of the sensor edge when a first portion of the stranded molecule is adhered to the edge portions of the at least one first electrode sheet and the at least one second electrode sheet of the at least one sensor as shown in FIG. 8D; and determining a characteristic of the first portion of the stranded molecule based on a detected first electrical signal. Interaction of the polymer with the first electrode sheet and the second electrode sheet is controlled by voltages applied to the global electrodes 301, 302 and by voltages applied to the first and second electrode sheets. In the example of FIGS. 8A-8H a negatively charged polymer 208 can be caused to adhere selectively to the edge electrodes by application of a positive charge to a particular edge 203 of FIGS. 8B and 8F or be repelled by a negative charge as shown in FIGS. 8C and 8E electrode 203. As shown in the sequence of FIGS. 8A through 8H by controlling voltages applied to global electrodes 301, 302 and sensor edge electrodes 203, 205 a polymer molecule can be caused to elongate and adhere segment by segment to edge electrodes 203, 205 for measurements of electrical properties and identification of polymer subunits in each segment. Examples herein include 2 (FIG. 8A-8H) or 3 (FIG. 4) edge sensor electrodes but the same construction and methods apply to a device with one or multiple edge sensor electrodes.

The methods, sensors, and devices described herein have many advantages over the prior technologies. For example, the multi-modal simultaneous sequencing approach can sequence nucleic acids by directly recording the tunneling conductance of nucleic acid bases along the molecular axis via parallel plane edge electrodes as shown in FIG. 5, and across a single base or base pair of a nucleic acid molecule via in-plane electrodes as shown in FIG. 6. The methods, sensors and devices described herein can sequence a nucleic acid molecule by also recording the ionic conductivity of the buffer solution around the nucleic acid molecule constrained by the edge. The detection of both the tunneling conductance of a nucleic acid base and the ionic conductivity of the buffer solution around the nucleic acid base are analyzed to chemically identify each subunit in turn. This multi-modal approach can allow the identification of post-synthesis modifications or other macromolecule modifications, including, for example, protein glycosylation, methylation, phosphorylation, and ubiquitination, by combining the molecular conductivity and ionic exclusion volume data. Due to the atomic thinness and the electric properties of the layers in the edge sensor, it can reduce the momentarily sampled base-pair or other monomer of the multimeric species number to as few as one, thus increasing the sequencing resolution.

In some embodiments, the sensor edge of the at least one sensor is functionalized with one or more chemical group(s) and the adhesion between the stranded molecule and the sensor edge of the at least one sensor occurs through electrostatic interaction, hydrogen bonding, hydrophobic interaction, intercalating interaction, ionic interaction, or a combination thereof between the stranded molecule and the chemical group(s).

The sensor edge including the exposed edge of the electrode sheet 203, 205 and/or the insulating layer 206 can be chemically modified or functionalized with a functional group. The terms “functionalized” or “functionalization” can refer to attaching, conjugating or grafting a moiety to a substrate (e.g., a graphene edge) with a functional group that is capable of reacting with an analyte. Functionalization of the edge with chemical moieties or enzymes that interact with specific target molecules (e.g., nucleotides or amino acids) can further enhance molecular interaction, periodically slow transit speed, and/or modulating the monitored electrical parameters as a biomolecule passes across the edge. For example, to enhance nucleotide-specific and/or amino acid-specific interactions, enzymes (can be, e.g., wildtype enzymes or modified enzymes) such as polymerases, exonuclease, proteases, helicases, or other chemical moieties can be introduced to the edge to specifically bind individual nucleotide or amino acid types or short sequences of polynucleotides or amino acids. In some embodiments, to contain the molecule/edge interaction DOF parameter field and increase control over molecule/edge interactions, a certain electrode edge functionalization can be beneficial. Introduced chemical groups can influence the affinity of the molecule (e.g., nucleic acid affinity) to the edge, charge transfer and extraction to/from the molecule (e.g., nucleic acid molecule), as well as contain the molecule in the optimal position with respect to the edge during measurement and translocation. Upon graphene lattice disturbance—breaking carbon-carbon bonds and exposing those unsaturated bonds to the environment (creating the edge via any means)—the exposed carbon atoms can become functionalized.

Various chemical edge modifications are suitable, including, for example, hydrogenation, hydroxylation, carboxylation, carbonylation, amination, and others identifiable to a person skilled in the art. Accordingly, the sensor edge (e.g., graphene edge) can be modified with a carbonyl, carboxyl, hydroxyl, aldehyde, carboxylate, ester, or amine functional group, or a combination thereof. The hydroxyl group can be a primary, secondary or tertiary hydroxyl group. The amine group can be a primary amine, secondary amine or tertiary amine. In some embodiments, hydrogenation of the edge of graphene electrodes can improve their sensitivity for nucleic acid sequencing purposes. Hydrogenation can introduce a hydrogen bond that can form between the hydrogen atom at the graphene electrode edges and atoms carrying a partial negative charge on the target molecule. The H-bonds between graphene electrodes and the translocating molecules (e.g., nucleic acid bases) can enhance the coupling between them and thus substantially increase the magnitudes of transverse tunneling currents. As a result, the current measurability and the speed with which the nucleic acid sequence can be read can be greatly improved. In some embodiments, the functionalization can cause an attractive force so that it can slow down the translocation of the molecule through the channel, providing more time for the transverse conductance measurement of each molecular unit located between the graphene edges.

These functional groups, possess varying chemical and physical properties with respect to molecular interaction and thus, in some embodiments, it is important to control the graphene edge chemistry with respect to utilities such as charge insertion/extraction steric interaction, electrostatic binding, hydrogen binding, hydrophobic/intercalating binding, metal modification, and biomolecule binding (e.g., enzyme binding). In an exemplary embodiment shown in FIG. 5, a p-phenylenediamine moiety 501, 502 functionalized to the graphene edge 203, 205 can be charge-conducting (conjugated), bear a slightly positive charge at neutral pH (base, pKa 6.2—binding the DNA phosphate group) and experience angular motion subject to electrical modulation (gating) by the other electrode potentially commensurate with single-base step translocation control. A variety of surface modifications and molecule interactions with edge sensors are discussed immediately below. Charge insertion/extraction.

In some embodiments, edges of a sensor, including electrode edges 203, 205 and/or edges of insulating layers 206, can be modified with certain charge-injecting groups to minimize the tunneling electrical resistance between the graphene edge and target molecule. Hydrogenation of the graphene edge is geometrically the smallest possible molecular functionalization (least effect on the interaction degrees of freedom field). It possesses good charge transfer properties, as well as the ability for hydrogen bonding due to a positive dipole moment. The edge can be modified with an organic molecule or an inorganic molecule. In some embodiments, and without being bound by any particular theory, if an organic molecule is selected for edge functionalization, it should be as electrically conductive as possible. Conjugated moieties can be strong candidates where the charge is conducted through their hybridized π-electron orbitals.

In some embodiments, as shown in FIG. 5, the backbone of a target molecule 208 can be positively or negatively charged. For example, the nucleic acid phosphate backbone is negatively charged at neutral pH and can be electrostatically attracted and bound by positively charged moieties around neutral pH such as amines, imine, and imidazolium salts. Therefore, in some embodiments, the edges can be modified with positively charged moieties such as amines, imine or imidazolium for nucleic acid sequencing. Phosphate backbone binding can be the preferred binding mode for edge read due to its nondiscriminatory (base-wise) nature, its outward positioning with respect to the molecular axis, as well as offering purely electronic binding control (electrode voltage modulation). One potential challenge is that the charge 504, if injected into the phosphate group, must tunnel 505 through the ribose unit prior to entering the delocalized π-electron cloud of the bases. This can be mitigated by increasing the tunneling bias as well as adding functionalizing moieties that would inject charge directly into the groove (major or minor for dsDNA) or couple to bases via hydrogen bonding described below.

In some embodiments, the target molecule can be bound to the edge structure at positions/sites other than phosphate backbone. Hydrogen bonding can also be utilized for measuring single-stranded DNA or even double-stranded DNA at some degree of intercalation.

The edge structure can be modified in a fashion that the electrostatic binding to the backbone (e.g., the phosphate group in a nucleic acid molecule) is coupled with additional hydrogen binding of conjugated moieties to the bases. In such way, the strong Coulombic interaction between the phosphate of nucleic acid molecule and hydrogenated or aminated edge can hold the nucleic acid molecule in the optimal position, while the conjugated moiety of appropriate geometry injects/extracts charge to/from the nucleic acid bases.

In some embodiments, the edge structure can be functionalized with intercalating agents such as DNA intercalating agents. DNA intercalating agents are generally conjugated hydrophobic heterocyclic ring molecules that resemble the ring structure of base pairs. Exemplary DNA intercalating agents include, for example, ammonified, SYBR Green, ethidium bromide, acridine orange, and actinomycin D. The DNA intercalating agents can intercalate between base-pairs via hydrophobic interactions and potentially provide greater tunneling conductivity from the electrode directly through the base π-stack than through the phosphate backbone (and ribose).

The edge can be functionalized with metal atoms/ions that can provide charge injection/extraction, nucleic acid affinity as well as a degree of non-voltage modulated electrostatic nucleic acid affinity in their ionic state. Exemplary metal atom/ion include, for example, Ca, Ni, Cu, Zn, Mg, K, and others identifiable to a person skilled in the art.

The one or more sensor edges described herein, including the edges of the at least one first electrode sheet 203, the at least one second electrode sheet 205, and the at least one first insulating layer 206 can be functionalized with a same functional group or different functional groups. For example, the edges of the at least one first electrode sheet and the at least one second electrode sheet can be functionalized with a same functional group or different functional groups. In some other embodiments, the edges of the insulating layers are functionalized with a same functional group as the electrode sheet. In some embodiments, the edges of the insulating layers are functionalized with a functional group different from the electrode sheet. In some embodiments, the edges of the insulating layers are not functionalized. In some embodiments, the one or more sensor edges described herein, including the edges of the at least one first electrode sheet, the at least one second electrode sheet, and the at least one first insulating layer can be functionalized with a same enzyme or different enzymes.

Provided herein also include systems for characterizing a molecule using one or more senor device described herein. The system can comprise one or more sensor devices/chips described herein, means for providing input electrical signals 304, means for reading out the electrical signals from the chips 305, and means 303, such as a computer and interface, for controlling input electrical signals and analyzing the electrical signals 305 to identify the analytes, e.g., base determination in the nucleic acid sequencing. In some embodiments, a system can comprise one or more sensor devices 100 described herein, an electronic control system 303 electrically connected 304, 305 to the one or more sensor device, the electronic control system configured to apply an input electrical control signal 304 to the one or more sensor devices and to receive an output electrical signal 305 from the one or more sensor devices when a stranded molecule 208 is adhered to the at least one sensor edge of the one or more sensor device, and a computer control system 303 for analyzing the output electrical signal from the one or more devices to determine a characteristic of the stranded molecule. The input electrical control signal can apply a voltage across the two or more global electrodes 301, 302, a voltage across the at least one first electrode sheet and the at least one second electrode sheet, or both. The input electrical control signal can comprise an alternating current voltage, a direct current voltage, an electrical impedance, or a combination thereof. The output electrical signal is detected 305 and can comprise a tunneling current between the at least one first electrode sheet and the at least one second electrode sheet, a voltage drop between the at least one first electrode sheet and the at least one second electrode sheet, and/or an ionic current flowing through the edge portions of the at least one first electrode sheet and the at least one second electrode sheet and an optical signal 307, 308. The computer control system can analyze the output electrical signal over time and use such information to determine a characteristic of the stranded molecule, for example, to identify the sequence of a nucleic acid molecule. The computer control system 303 can also include components for computational data analysis or the acquired signal 305 can be analyzed remotely from the computer control system. In the embodiments of nucleic acid sequencing, the electronic signals from the chips processing nucleic acid molecules represent raw data attributed to the conductivity and exclusion volume of nucleic acid bases. Each datapoint can represent a signal originating from one or more bases positioned between the edge electrodes. The computational data analysis component of the computer control system can convert raw data attributed to the conductivity and exclusion volume of the nucleic acid bases to the identity of the nucleic acid bases. The computer can also control the performance of the one or more sensor devices, for example, by providing a sequence of input electrical signals to the global electrodes and/or the first and second electrode sheets in the sensor device. The systems described herein can be used in connection with various operating systems to execute algorithms or computer-implemented instructions designed to translocate the molecule in a controlled manner and to identify the characteristic of individual monomers in a stranded monomer such as to identify the sequence on individual bases in a nucleic acid molecule.

In some embodiments, detecting an electrical signal also comprises measuring a tunneling current between the two closely spaced electrode sheets. The two closely spaced electrode sheets can be two parallel plane electrodes vertically aligned such that one electrode sheet is positioned on the top of the other as for electrode edges 203, 205. FIG. 5 illustrates an exemplary embodiment of tunneling conductance measurement in a nucleic acid molecule along the molecular axis. Once the nucleic acid molecule is in contact with the edge electrodes in either stretched or relaxed state and is bound to the edge moieties, its electrical properties are measured. Graphene electrodes are voltage biased (e.g., 0.1V) and charge is injected into the nucleic acid molecule via edge moieties (e.g., phenylenediamine) 501 at the top graphene electrode. The electrons tunnel along the π-stacking of the nucleic acid bases (e.g., AAC) and are ejected into bottom graphene electrode. The tunneling current 506 is recorded for the AAC base sequence. After the nucleic acid molecule is translocated 507 along the edge by one base pair the tunneling current measurement is repeated. In this position, the tunneling conductivity of TAA base sequence is then recorded. Computational algorithms can be used to compare the tunneling conductance data obtained from the sequentially translocated nucleic acid molecule and to elucidate its base pair sequence. In some embodiments, detection of an electrical signal is a combined measurement of excluded volume of ions in the opening around the molecule and a tunneling conductance of units between the two closely spaced electrodes. In some of these embodiments 600, the molecule 208 (FIG. 6) can be surrounded by the sensor edge in all directions.

In some embodiments, detecting an electrical signal can further comprise detecting an in-plane tunneling conductance 601 across a molecule or a portion thereof. FIG. 6 illustrates an exemplary embodiment of in-plane tunneling conductance measurement in a nucleic acid molecule. Variations in the in-plane current through a graphene electrode due to the traversal of a molecule can be measured. The aperture or gap 602 in the graphene electrode is small enough to ensure that the nucleotide under interrogation can bridge the two electrical contacts 603, 604. In some embodiments, the nucleic acid molecule under interrogation is fully enclosed by the edge of the graphene electrode 603, 604. In some embodiments, the aperture or gap 602 can be a nanopore 605 through which a molecule 208 can translocate. Two walls of the nanopore are electrodes 603, 604 and two walls of the nanopore 605 are dielectric 606.

In some embodiments, detecting the first electrical signal and/or the second electrical signal comprises applying a voltage between the at least one first electrode sheet and the at least one second electrode sheet and measuring a tunneling current between the at least one first electrode sheet and the at least one second electrode sheet. In some embodiments, detecting the first electrical signal and/or the second electrical signal can comprise applying a voltage across the global electrodes 301, 302 (see FIG. 8) and measuring a voltage drop between the at least one first electrode sheet 203 and the at least one second electrode sheet 205. In some embodiments, detecting the first electrical signal and/or the second electrical signal comprises measuring an ionic current flowing through the edge portions of the at least one first electrode sheet and the at least one second electrode sheet.

In some embodiments, the input electrical signal 304 applies a voltage across the two or more global electrodes, a voltage across the at least one first electrode sheet and the at least one second electrode sheet, or both. In some embodiments, the input electrical signal 304 comprises an alternating current voltage, a direct current voltage, an electrical impedance, or a combination thereof. In some embodiments, the output electrical signal 305 comprises a tunneling current between the at least one first electrode sheet and the at least one second electrode sheet, a voltage drop between the at least one first electrode sheet and the at least one second electrode sheet, an ionic current flowing through the edge portions of the at least one first electrode sheet and the at least one second electrode sheet, and as shown in FIG. 6, an in-plane tunneling conductance across the stranded molecule through a pair of electrodes 603, 604 in the same plane, or a combination thereof. Some embodiments include electrodes 203, 205 that are stacked upon one another as well as electrodes 603 and 604 located in the same plane. Each of the latter electrodes 603, 604 can also include a second set of in plane electrodes stacked vertically upon one another as shown in FIG. 2 for electrodes 203, 205.

A notable advantage of some embodiments of the edge read technology provided herein is in electronic control of the speed and direction of molecular translocation. For example, an array of individually controlled electrodes (e.g., two buffer-biasing electrodes and two/three/more graphene edge DNA-biasing electrodes) when voltage modulated in certain sequence, can ensure base-by-base nucleic acid 208 translocation with prescribed frequency. The translocation directionality 701 can be controlled electronically via such voltage modulation thus allowing to “re-wind” 703 and re-sequence the nucleic acid 704 molecule as desired.

One of the major current solid-state nanopore nucleic acid sequencing challenges is the fast nucleic acid translocation speed through the active region of the device. Most often, the nucleic acid translocation speed is controlled enzymatically, e.g., DNA is “fed” via an enzyme with precise translocation step-size and controllable step frequency. In the devices described herein, certain materials having high nucleic acid affinity due to their surface or bulk properties can be used to slow the translocation 703 of the nucleic acids. The surface of the channel 103 leading to the edge (e.g., the lateral channel) can be modified as shown in such that is can either attract or repel nucleic acid molecules. For example, the interior surface of the lateral channel 103 can be made of glass for nucleic acid repulsion or aluminum oxide for nucleic acid affinity. The interior surface of the channel can also be chemically modified to comprise organic molecules (SAMs, lipid bilayers, etc.) such as phenyl or amino groups. The interior surface of the channel can also be chemically modified with a biomolecule.

In some embodiments, to slow down a nucleic acid translocation, the channel 103 can be filled with a hydrogel such as agarose, polyacrylamide, PVA, or others identifiable to a skilled person. The hydrogel can typically slow down DNA by orders of magnitude depending on the electrical bias, DNA length and the gel density. The hydrogel can be naturally occurring or synthetic. For example, unmodified 5% agarose gel has a pore size on the order of 30 nm, while 1% agarose gel has a pore size of about 200-300 nm. By adjusting the gel density, the translocation speed of a biomolecule can be controlled. Gels of certain density can be further chemically modified with affinity sites such as phenyl and/or amino groups to slow down molecular movement. In some embodiments, the channel 103 can be filled up with nanoparticle matrices which can slow down nucleic acid translocation with similar velocity effects. Adding control to the speed of translocation (including reversibility) across an edge formed by the 2-D electrode material (e.g., graphene) disclosed herein or across the device can allow the device to dwell on a particular sequence until a level of certainty of the identity of a molecular unit (e.g., a base) in achieved or more simply to control the translocation speed to a speed which is optimal for monomer or multimer identification and/or instrument throughput.

In some embodiments, the method comprises: translocating the stranded molecule across the sensor edge; and measuring a second electrical signal 305 through the edges when a second portion of the strand molecule is adhered to both edge portions of the at least one first electrode sheet and the at least one second electrode sheet, following the translocation of the stranded molecule, acquiring a third electrical signal following another translocation of the molecule 208 and so forth. In some embodiments, the method comprises: linearizing the stranded molecule along the fluidic passage, e.g., by adjusting the magnitude and direction of the voltage applied to global electrodes 301, 302 and/or the edge electrodes 203, 205 across the fluidic passage. In some embodiments, the first portion of the stranded molecule is in a stretched conformation when the first portion of the stranded molecule is adhered to both edge portions of the at least one first electrode sheet and the at least one second electrode sheet of the at least one sensor as shown in FIG. 8A, not stretched and FIG. 8B stretched after applying voltages across global electrode 301 and edge electrode 205. FIG. 8A-8H illustrate a non-limiting, exemplary workflow of sequencing a molecule 208 using the method and device described herein. In FIG. 8A, a prepared sample 208 is deposited into the loading well 101. The electrophoretic movement of a nucleic acid molecule through the wedge channel is encouraged by energizing global electrode 301 with appropriate voltage bias (e.g., 0.5V). The wedge channel is configured such that only one molecule is allowed into the channel due to electronic, entropic, and steric intermolecular competitive interactions. As the molecule enters the channel, the molecule stretches linearly (FIG. 8B) and translocates along the channel 103 towards the edge bottom electrode (302) and through the channel opening 201. The leading end of the molecule is electrostatically attracted to and adheres to edge electrode 205 via edge moiety interaction aided by an adhesion force that can attribute to various physical and/or chemical interactions between the molecule and functional groups on the edge 205 (FIG. 8B). Electrostatic stretching of the nucleic acid molecule can be achieved by energizing electrodes 301 (+) vs 302(−) and 203(−) with appropriate voltage bias (e.g., 0.2 V) (FIG. 8C). The selected voltage applies an electrostatic force onto the for example nucleic acid molecule 208 with a magnitude less than the adhesion force at the bottom electrode sheet 205. The nucleic acid molecule remains adhered at the edge electrode 205 and stretches across the sensor edge towards the loading well 101 (FIG. 8C). Once the nucleic acid molecule is in a stretched conformation, the voltage bias at global electrode 301 is released and the molecule adheres to edge electrodes 203, 205 with an adhesion force that can attribute to various physical and/or chemical interactions between the molecule and functional groups on the edge 203, 205 (FIG. 8D). It is noted that in a relaxed state, the number of bases between edge electrodes 203, 205 is n, which is determined by dielectric thickness 206 and moiety identity, while in a stretched state the number of bases between edge electrodes 203, 205 is n−1. Measurement of DNA tunneling conductance between edge electrodes 203, 205 can thus be performed and base-sequence conductance profile can be recorded 305. Either 2-point measurement or 4-point measurement can be performed. For the 2-point measurement, a voltage bias between global electrodes 301, 302 is introduced (e.g., 0.1V) and the resulting current is measured. For the 4-point measurement, a voltage bias between edge electrodes 203, 205 is introduced (e.g., 0.1V) and the voltage drop between edge electrodes 203, 205 is measured. The voltage drop essentially records the resistivity profile of base-sequence between the edge electrodes. In some embodiments, a 4-point probe measurement can be preferrable as it is not influenced by the contact resistance. In such cases, there is no charge transfer between the electrodes and the DNA molecule and only the voltage drop is measured. In addition to the tunneling conductance measurement, measurement of the ionic flow around the edge can also be performed and the ionic exclusion volume profile of the nucleic bases at the sensor edge can be recorded. A combined tunneling and ionic flow measurement is thus performed.

Once the conductance and/or resistance profile is recorded, the nucleic acid molecule can be translocated across the sensor edge by one base. To achieve this goal, the tail end of the nucleic acid molecule is decoupled from the edge 203 by energizing 203(−) vs global electrode 302(+) with a voltage bias such that the adhesion force imposed at 203 is overcome, and the nucleic acid tail end relaxes while being anchored at second edge electrode 205 via the adhesion force (FIG. 8E). Once the molecule is in the relaxed state, a positive voltage bias at edge electrode 203 is introduced and the molecule adheres to edge electrode 205 with an adhesion force (FIG. 8F). A conductance and/or resistance measurement can be performed at this stage to confirm the state of the nucleic acid molecule. The nucleic acid molecule 208 can be stretched out towards the global electrode 302 by a voltage bias between global electrode 302 (+) and edge electrode 205(−) (FIG. 8G). Once the molecule is stretched out, the voltage bias at global electrode 301 (−) is reversed and the molecule adheres to edge electrode 205 (+) with an adhesion force (FIG. 8H) and the molecule is advanced by one base as compared to in FIG. 8D. The stretching motion mechanism disclosed herein can also be used to reverse the molecule translocation at any point during sequencing. To sequence the entire molecule, the above steps can be repeated as needed until the last base reaches edge electrodes 203, 205. Once the entire molecule is sequenced, the molecule is detached from the edge electrodes via strong negative bias between global electrodes 301(−), 302 (−) and edge electrode 205 (+) and exits through the vertical channel 107. Another molecule can at this point enter the lateral channel 103 to be sequenced. The described modulation should be performed at frequencies and waveforms commensurate with nucleic acid translocation velocity, electrode interaction dynamics and electric field strength distribution as will be apparent to a person skilled in the art.

The device can also be pre-loaded at the loading well 101 with components to enhance the entry of a particular class or subset of biomolecules across the edge, such as, for example a complementary oligonucleotide or an antibody, or to deplete a certain class of biomolecules, such as, for example, by adding proteinase K to digest proteins or an oligonucleotide distant from the sensor to deplete ribosomal RNAs. Methods for Molecular Characterization

Disclosed herein also include methods of characterizing a stranded molecule. In some embodiments, the method can comprise providing a sensor device 100 (FIGS. 1-8) described herein, applying a voltage across the fluidic passage to produce a current flow through the fluidic passage 103, 107, causing a single stranded polymer molecule 208 to translocate through the fluidic passage at a time and allowing said single stranded polymer molecule to interact with the sensor edge of the sensor 203, 205, detecting a first electrical signal between the at least one first electrode sheet 203 and the at least one second electrode sheet 205 of a sensor edge when a first portion of the stranded polymer molecule is adhered to the edge portions of the at least one first electrode sheet and the at least one second electrode sheet of a sensor, and determining a characteristic of the first portion of the stranded molecule based on the detected first electrical signal. Then translocating the molecule 208 as described above and then obtaining a second electrical signal between edge electrodes 203, 205, translocating the molecule 208 again, obtaining a third electrical signal and so forth. In some embodiments the device 100 further includes electrodes 603, 604 and electrical signals are obtained both along the length of molecule 208 and across molecule 208 as shown in FIG. 6 for each translocation and measurement step. The stranded molecule 208 can be partially or fully surrounded by the sensor edge.

The method and device described herein can be compatible with a range of biomolecules that are polymeric in nature with unit repeat structures. A stranded molecule to be characterized can be a polymer, a polynucleotide, a polysaccharide, a polypeptide, or a combination thereof. In some embodiments, the stranded molecule is a polynucleotide such as DNA or RNA. The polynucleotide can be naturally occurring or synthetic, single-stranded or double-stranded.

The method and device described herein are capable of determining a range of characteristic of a biomolecule, so long as the characteristic of a biomolecule affects the electrical signal (e.g., tunneling current, ionic current, resistance, impedance, voltage) between the two closely spaced electrode sheets when the biomolecule 208 is in close vicinity of or adhered to the edges of the electrode sheets 408, 409, 410 and in some embodiments parallel sheets 603, 604. For example, a characteristic of a portion or a chemical unit of a stranded molecule 208 can include, e.g., nucleic acid or other polymer base sequence, chemical modification such as methylation, phosphorylation, glycosylation, ubiquitination, lipidation, proteolysis, PEGylation, amino acid sequence, biomolecule secondary structure, a binding event such as a protein-polynucleotide binding event, or others identifiable to a person skilled in the art. In cases the stranded molecule is a nucleic acid molecule (e.g., DNA or RNA), determining the characteristic of the stranded molecule can comprise determining base sequence information of the nucleic acid. Referring to FIGS. 8A-8H, the method can further comprise translocating (FIGS. 8E-8G) the stranded molecule 208 across the sensor edge 203, 205, and measuring a second electrical signal through the edges when a second portion of the stranded molecule is adhered to both edge portions of the at least one first electrode sheet and the at least one second electrode sheet (See FIGS. 8G and 8H), following the translocation of the stranded molecule. Each portion of the stranded molecule can correspond to a single chemical unit (e.g., one base of a nucleic acid molecule or one amino acid of a polypeptide). In some embodiments, each portion of the stranded molecule can comprise more than one chemical unit (e.g., one, two, three, four, five or more units). The number of units to be characterized in an individual detection depends on the thickness 204 of the edge geometry. For example, in the case of a nucleic acid, five bases can fit at the edge electrode composed of graphene/3-layer hBN/graphene. As the stranded molecule translocates through the fluidic passage, additional electrical signals can be measured as each portion of the stranded molecule (e.g., each base of a nucleic acid molecule) sequentially adheres to the edges of the two closely spaced electrode sheets. Therefore, the method can further comprise measuring a third electrical signal through the edges when a third portion of the stranded molecule is adhered to both edge portions of the at least one first graphene sheet and the at least one second graphene sheet. The method can further comprise measuring a fourth electrical signal through the edges when a fourth portion of the stranded molecule is adhered to both edge portions of the at least one first graphene sheet and the at least one second graphene sheet. The measurement can be continued till the terminal unit of the stranded molecule adheres to the electrode edges.

The method can further comprise linearizing the stranded molecule as the molecule translocates through the fluidic passage. The stranded molecule can be linearized by adjusting the magnitude and sign of the voltages across the fluidic passage, such as by adjusting the magnitude and sign of the voltage across the global electrodes 301, 302 (see FIGS. 8A and 8B) and/or the two closely spaced electrode sheets 203, 205 (e.g., graphene sheets). Other methods and structures can also be used to facilitate the linearization of the molecule, including for example chemical modification of the interior surface of the fluidic passage 103, 107, or using a variety of obstacles or other structural confinements.

The stranded molecule can be in a stretched conformation (see FIG. 8B) or in a relaxed, flexible conformation as the molecule translocates through the channel. For example, the first portion of the stranded molecule 208 can be in a stretched conformation (FIG. 8B) when the first portion of the stranded molecule is adhered to both edge portions of the at least one first electrode sheet and the at least one second electrode sheet of the sensor. The adhesion of the stranded molecule to the sensor edge can occur through physical and/or chemical interactions between the stranded molecule and the sensor edge. The sensor edge 203, 205 can be functionalized, as shown in FIG. 5, with one or more chemical group(s) or functional group(s) and the adhesion between the stranded molecule and the sensor edge can occur through electrostatic interaction, hydrogen bonding, hydrophobic interaction, intercalating interaction, ionic interaction, or a combination thereof between the stranded molecule and the chemical group(s). For example, as shown in FIG. 5, the exposed edges of graphene sheets can be functionalized by attaching a chemical moiety 501 to the edges which has an affinity to a portion of the biomolecule 208.

Detection of an electrical signal can take place when the stranded molecule is in close vicinity of and/or adhered to the sensor edge. The particular electrical signal depends on the context in which the method and device are employed as well as the device configuration. The electrical signal to be detected can include, for example, a tunneling current across the two closely spaced electrode sheets, a voltage drop between the two closely spaced electrode sheets, an ionic current flowing through the sensor edge, resistance, impedance, electric potential, translocation time or transit speed of the molecule through the channel.

In some embodiments, detecting an electrical signal comprises measuring an ionic current flowing through the edge portions of the two closely spaced electrode sheets. in some embodiments, as a molecule is driven across the sensor edge by an electric field, it excludes ions in the opening around the sensor edge, resulting in a temporal decrease in the ionic current 505. The magnitude and the duration of the current blockade provide information on the diameter and length of the molecule, respectively. For sequencing, each nucleotide blocks the ionic current in a unique way that is dependent on its molecular size and shape.

Following the detection, the stranded molecule can advance (FIGS. 8E-8F) along the passage 107 by a segment or a portion of the molecule which can comprise one or more chemical units. Therefore, translocating the stranded molecule can move the stranded molecule across the sensor edge by a portion or a unit of the stranded molecule (e.g., a base). Translocating the stranded molecule can comprise dissociating (FIG. 8E) the first portion of the stranded molecule from the edge portions of the at least one first electrode sheet and the at least one second electrode sheet, and adhering the second portion of the stranded molecule to the edge portions of the at least one first electrode sheet or the at least one second electrode sheet (see FIGS. 8G, 8H). Dissociating the first portion of the stranded molecule from the edge portions can comprise dissociating the first portion of the stranded molecule from the edge portion of the at least one first electrode (FIG. 8E), followed by dissociating the first portion of the stranded molecule from the edge portion of the at least one second electrode (FIG. 8G). During the dissociation step, the stranded molecule can adopt a relaxed, flexible conformation, and the stranded molecule can remain adhered to at least one edge portion of the at least one first electrode sheet or the at least one second electrode sheet. Dissociating the first portion of the stranded molecule and adhering the second portion of the stranded molecule can be achieved by (1) adjusting the magnitude and/or direction of the voltage between a global electrode 301, 302 and the at least one first electrode sheet 203 or the at least one second electrode sheet 205, (2) adjusting the interaction between the edge portions and the stranded molecule, or both. For example, dissociating the first portion of the stranded molecule and adhering the second portion of the stranded molecule can comprise independently energizing the at least one first electrode sheet or the at least one second electrode sheet.

In some embodiments, it can be beneficial for better translocation control and measurement accuracy to introduce a third electrode sheet and a second insulating layer into the stack (see, for example, FIG. 4). The third electrode sheet 409 can comprise any atomically thin, electrically conductive material described herein or known in the art. In some embodiments, the first 408 and second 410 electrode sheets comprise graphene and the third electrode sheet 409 does not comprise graphene. In some embodiments, all the edge electrode sheets are graphene sheets.

At high modulation frequencies (e.g., MHz) the global electrodes alone may be inadequate to control movement of the stranded polymer molecule 208 due to buffer-related capacitive effects, whereas within the nanoscopic geometry of the edge stack 408-410 (well within the Debye length of expected working ionic strength buffers) such high frequency modulations should be allowable. With three edge electrodes 1 nm apart, a nucleic acid molecule, for example, can be moved locally, relying solely on the edge electrodes in the “caterpillar” fashion as shown in FIG. 4. In some embodiments, the degree of control of such system can be significantly higher than the one also involving liquid-biasing electrodes for nucleic acid “stepping”. In some embodiments, and without being bound by any particular theory, this is because the electrostatic interaction force between the nucleic acid molecule and the edge electrodes is independent of the degree of advancement of the nucleic acid molecule with respect to the sensor edge towards either global electrode 301 or global electrode 302. The edge electrodes can provide local, nanoscopic electric field gradient acting on individual phosphate backbone units in the vicinity of the sensor edge. While the electrostatic force imposed of the nucleic acid molecule by the global 301, 302 electrodes can change depending on the degree of advancement/translocation across the sensor edge and thus the degree of advancement will have to be accounted for during translocation control of this kind.

### Sensor Device Fabrication

An exemplary, non-limiting wafer-scale fabrication procedure for the edge sensor device 100 as already described above, is illustrated in FIG. 9. The procedure includes the underlined process steps 901-912 and reference numerals to the structures formed 913-923 and references to structures described above in FIGS. 1-3 . A carrier substrate 108 e.g., silicon wafer with oxide layer deposited thereon is formed 901. Metal electrodes/traces 913 for contacting the bottom edge electrode 203 are deposited 902 on the substrate. A layer of edge electrode material 914 (e.g., monolayer graphene) is then deposited over the entire carrier surface including the metal electrodes/traces such that graphene makes electrical contact with the metal electrodes/traces 913. A stack of dielectric materials 915, as an example, 3 layers hBN 206 in FIG. 2, is deposited 903 on top of the edge electrode material covering the entire carrier surface, including the graphene layer 914. Another set of metal electrodes/traces 916 are deposited 904 on top of the dielectric layer 915 followed by depositing 905 a second layer of electrode material 917 and dielectric material 918. Steps 904 and 905 can be repeated to create a three electrode edge sensor as shown in FIG. 4 and again to make a four electrode sensor and so on. Steps 904 and 905 can be omitted to produce a single edge electrode device. The process steps as shown in FIG. 9 will produce a two edge electrode device as shown above in FIG. 2. In some embodiments, the first conductor layer 913 and/or the second conductor layer 916 comprises a metal pad. In some embodiments, the metal pad is a Ti/Au pad or a Cr/Au pad. In some embodiments, depositing one or more dielectric layer comprises sequentially depositing a first dielectric layer and a second dielectric layer. In some embodiments, the first and second dielectric layers comprise a same dielectric material or different dielectric materials. In some embodiments, the first and second dielectric layers are different in thickness.

A top dielectric layer 919 is deposited 906 over the entire surface, e.g. 10 nm SiN, to passivate. The top dielectric layer 919, the dielectric layers between the edge electrodes 915, 918 and electrode layers 914, 917 are all patterned 907 into a tapered lateral fluidic channel 921 (also labeled 103 of FIG. 1). All layers are patterned simultaneously using for example reactive ion etching (RIE) such that the wedge shaped edge electrodes and intervening dielectric layers overlap exactly. The vertical fluidic channel 922 (also item 107 of FIG. 1) is etched through all the layers including the substrate such that it intersects the lateral fluidic channel at its narrow end, and, both the lateral and vertical fluidic channels are joined 908. Etching the vertical channel exposes the edge electrodes 203, 205 just under the nanoscopic opening in the lateral fluidic channel, thus allowing electrical manipulation and investigation of the nature of a molecule translocating through the lateral and vertical channels as already discussed above. The location of the vertical channel 920 along the length 926 of the wedge shaped lateral channel determines the width 202 of the edge electrodes exposed in the vertical channel (see also items 209, 202 of FIG. 2). The vertical channel through the edge stack films is opened via a controlled dielectric breakdown process. The process is stochastic and relies on creating a voltage across the edge stack membrane to the point where the dielectric material in the membrane experiences electric breakdown and the charge flows across the membrane along the breakdown path creating the aperture 922 in the membrane in the breakdown point and growing the aperture as the charges progress through it over time. The aperture size 107 can thus be controlled via controlling the pulse current and frequency parameters as well as its displacement 209 along the lateral fluidic channel as already discussed. The apertures created this way can be as small as 1 nm in diameter and can extend through membranes that are hundreds of nanometers thick. A capping layer 923, e.g., 100-micrometer thick glass with prepatterned openings (e.g., loading wells 924) and metal electrodes (global electrophoresis electrodes), are wafer-bonded on top of the entire carrier such that the loading wells 101 is aligned with the wide end of the horizontal channel (see, for example, FIG. 1) 909. Macroscopic loading area 924 and an exit chamber 925 are finally added 910, 911.

Referring now to FIG. 10, in some embodiments, the sensor device is a sensor chip. In some embodiments, the sensor device comprises a plurality sensors each exposed to a distinct polymer loading passage 1004. In some embodiments, each of the sensor device comprises: a loading well 101 and a fluidic reservoir 1004 in fluidic connection to the fluidic passage 103, wherein the loading well provides an inlet to the fluidic passage and the fluidic reservoir provides an outlet from the fluidic passage. In some embodiments, (a) multiple electrodes and/or multiple insulating layers are stacked on top of each other; or (b) multiple independent side by side devices are formed. As illustrated in FIG. 10, to fabricate a sensor device described herein, edge stack materials (e.g., graphene/3-layer hBN/graphene) can be supported with a thin film of aluminum about 200 nanometers thick 1001. The aluminum can be deposited onto the edge materials stack that has previously been transferred onto the receiving substrate. Alternatively, the aluminum can be deposited onto the edge materials stack while still on graphene/hbn/graphene synthesis substrate (e.g., copper, wafer, etc.). Metal assisted exfoliation (MAE) can then be used to remove the edge stack materials from the synthesis substrate and onto a receiving substrate. Additional information about metal assisted exfoliation process is described, for example, in U.S. Pat. No. 9,840,024. The receiving substrate can be a thin sheet material (e.g., glass, polymer, ceramic, metal, with a thickness 1006 of about 10 micrometers to millimeters) with small openings (e.g., microwells) 1005 similar to the loading well 101 in the wedge format, in a diameter of about 10 micrometers. The edge stack materials can be patterned into desired electrode shapes and addressed with metal electrodes/traces prior to aluminum deposition. The edge stack/aluminum/perforated sheet support is then anodized in oxalic acid with the aluminum being the anode. Upon oxidation, the aluminum converts to aluminum oxide and a hexagonal array of nanochannels forms open on the side opposite to the edge stack materials (see FIG. 10, step 1003). The anodization only occurs within the perimeter of the microwell where oxalic acid is in contact with the aluminum film and forms the channels open into the microwell. The channel density can be controllable with anodization parameters including for example temperature, voltage, reagent concentration, and others identifiable to a person skilled in the art. The channel diameter 1007 is between 20 nm and several hundred nanometers and can also be controlled. There is a layer of aluminum oxide 30 nm thick that forms on the edge materials stack interface. The closed-loop edge opening can then be created through the edge stack supported with the 300 nm aluminum oxide via dielectric breakdown process. The dielectric breakdown process is stochastic and forms only one opening per microwell regardless of the number of nanochannels interfacing the edge membrane. Each microwell can have an individual liquid-biasing electrode in it as in global electrode 301 (see FIG. 3) such that the voltage bias across the membrane in all wells 1005 individually can be controlled (which is not required for anodization but required for the breakdown process). The electrode can be patterned on either side of the microwell sheet (around the perimeter of the well, for example) or be located inside the microwell. The density of edge sensors in this case will be determined by the density of the microwells.

In summary, the method comprises: providing a stack film comprising at least one first electrode sheet, at least one first insulating layer and at least one second electrode sheet, the stack being deposited with a metal film and with a substrate comprising one or more microwells 1005 in connection with the metal film; anodizing the metal film in an electrolyte solution, thereby converting the metal to metal oxide and forming a plurality of nanochannels in the metal film; and forming an aperture through the stack film in a microwell by applying a voltage across the stack film. The microwell becoming the vertical fluidic channel for each of a plurality of sensor devices. In some embodiments, the metal film comprises aluminum, magnesium, titanium, or a combination thereof. In some embodiments, the metal film is an aluminum film. In some embodiments, the electrolyte solution is an oxalic acid. In some embodiments, the density and/or dimension of the plurality of nanochannels are controlled by adjusting the voltage and temperature of the anodization and/or the concentration of the electrolyte solution. In some embodiments, the method further comprises: positioning a global electrode in each of the one or more microwells. In some embodiments, the aperture has a dimension of about 1 nm-5 nm. In some embodiments, the plurality of nanochannels have a dimension of 20 nm to 100 nm

### SUMMARY

A device for characterization of polymers is described. The device includes a plurality of atomically thin electrodes with their edges exposed along a fluidic channel through which a single stranded polymer molecule is controllably positioned. The polymer includes a series of subunits where each in sequence is identified by an electrical signal between two of the electrodes when in contact with the two electrodes. The position of the polymer strand within the fluidic channel is controlled by voltages applied to the atomically thin electrodes as well as global electrodes located within the fluidic channel. The electrical signal includes tunneling currents along the length of each segment, ionic currents within solutions containing the polymer segments, and, in some embodiments tunneling currents across each polymer segment, and, based upon those measurements, identifying the chemical composition of each segment and therefore the chemical composition of the polymer as a sequence of subunits. The system and techniques are applicable to a wide range of polymers. A typical use is the base sequence of nucleic acid including DNA and RNA without a need for derivatization of the nucleic acid.

What is claimed is:

1) A sensor device (100) for characterizing a stranded molecule (208), the sensor device comprising:
a) a first electrode sheet (914), a second electrode sheet (918), and an insulating layer (915) disposed between the first electrode sheet and the second electrode sheet,
b) the first electrode sheet, the second electrode sheet and the insulating layer are atomically thin and etched to form stacked and tapered electrodes (921) coincident with and lying beneath a lateral tapered channel (103), and,
c) the lateral tapered channel is connected to a vertical fluidic channel (107) at a smaller end of the tapered lateral fluidic channel, and an edge of each of the tapered electrodes (203, 205) and an edge of the insulating layer (206) are exposed to the vertical fluidic channel thereby capable of interacting with the stranded molecule in the vertical fluidic channel, and measuring an electrical current (506) along a length of a segment of the stranded molecule held between the exposed edge of the first electrode edge (203) and of the second electrode (205).

2) The sensor device of claim 1, wherein the first electrode sheet (203) and the second electrode sheet (205) are each atomically thin sheets of graphene and the insulating layer (206) is one or more sheets of atomically thin boron nitride or hexagonal boron nitride.

3) The sensor of claim 1, wherein the exposed edge of the first electrode sheet (203) and the exposed edge of the second electrode sheet (205) are each functionalized with p-phenylenediamine (501).

4) The sensor device of claim 1 further comprising a first global electrode (301) located in
the lateral fluidic channel and a second global electrode (302) located in the vertical fluidic channel and a computer control (303) controlling a power supply (304) to apply a voltage to the tapered electrodes (203, 205) and the global electrodes (301, 302), and, a signal acquisition system (305) that measures voltages and currents between the edges of each of the tapered electrodes (203, 205) and between the global electrodes (301, 302), and, the power applied to the global electrodes and the edge electrodes controllably moves the stranded molecule electrophoretically through the lateral tapered channel and the vertical channel and along the edge electrodes segment by segment of the stranded molecule to measure the current through the length of each segment of the stranded molecule along the length of the stranded molecule and the current is analyzed to identify the chemical structure of each segment of the stranded molecule.

5) A multilayer electrode comprising:
a) a silicon base
b) a first dielectric layer deposited upon the silicon base (901),
c) a plurality of electrode layers each stacked upon one another and deposited upon the first dielectric layer, each of the plurality of electrode layers comprising a layer of graphene (914) and a layer of a second dielectric (915) and a metallic electrical contact (913),
d) wherein an edge of each of the graphene layers (203) can be contacted, and, wherein each of the metallic electrical contacts can be individually electrically addressed.

6) The multilayer electrode of claim 5 wherein the layer of graphene in each of the plurality of electrode layers is atomically thin.

7) The multilayer electrode of claim 5 wherein the layer of the second dielectric in each of
the plurality of electrode layers is atomically thin boron nitride or hexagonal boron nitride.
